(19) United States
(12) Patent Application Publication
(10) Pub. No.: US 2005/0014753 A1
(43) Pub. Date: Jan. 20, 2005

(54) Novel compounds and compositions as protein kinase inhibitors

(75) Inventors: Qiang Ding, San Diego, CA (US); Taebo Sim, San Diego, CA (US); Guobao Zhang, San Diego, CA (US); Francisco Adrian, San Diego, CA (US); Nathanael S. Gray, San Diego, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: IRM LLC, Hamilton HM LX (BM)

(21) Appl. No.: 10/817,328

(22) Filed: Apr. 1, 2004

Related U.S. Application Data
(60) Provisional application No. 60/460,838, filed on Apr. 4, 2003.

(51) Int. Cl. A61K 31/53; A61K 31/505

(52) U.S. Cl. 514/241; 514/256; 514/340; 544/209; 544/319; 544/326

(57) ABSTRACT

The invention provides a novel class of compounds, pharmaceutical compositions comprising such compounds and methods of using such compounds to treat or prevent diseases or disorders associated with abnormal or deregulated tyrosine kinase activity, particularly diseases associated with the activity of PDGF-R, c-Kit and Bcr-abl.

### CROSS-REFERENCES TO RELATED APPLICATIONS

This application claims benefit of U.S. Provisional Application No. 60/460,838, filed Apr. 4, 2003, which application is incorporated herein by reference for all purposes.

### BACKGROUND OF THE INVENTION

1. Field of the Invention

The invention provides a novel class of compounds, pharmaceutical compositions comprising such compounds and methods of using such compounds to treat or prevent diseases or disorders associated with abnormal or deregulated tyrosine kinase activity, particularly diseases associated with the activity of PDGF-R, c-Kit and Bcr-abl.

2. Background

The protein kinases represent a large family of proteins, which play a central role in the regulation of a wide variety of cellular processes and maintaining control over cellular function. These kinases include receptor tyrosine kinases, such as platelet-derived growth factor receptor kinase (PDGF-R), the receptor kinase for stem cell factor, c-Kit, and non-receptor tyrosine kinases, such as the fusion kinase Bcr-abl.

Chronic myeloid leukemia (CML) is an extensively studied human cancer that is caused by a reciprocal translocation that fuses the Abl proto-oncogene on chromosome 9 with a gene on chromosome 22 called Bcr. The resulting fusion protein Bcr-abl is capable of transforming B-cells by increasing mitogenic activity, reducing sensitivity to apoptosis and altering the adhesion and homing of CML progenitor cells. STI-571 (Gleevec) is an inhibitor of the oncogenic Bcr-abl tyrosine kinase and is used for the treatment of chronic myeloid leukemia (CML). However, some patients in the blast crisis stage of CML are resistant to STI-571 due to mutations in the Bcr-abl kinase.

The novel compounds of this invention inhibit one or more kinases; in particular wild type and one or more of the mutant forms of Bcr-abl and are, therefore, useful in the treatment of kinase-associated diseases, particularly Bcr-abl kinase associated diseases.

### BRIEF SUMMARY OF THE INVENTION

In one aspect, the present invention provides compounds of Formula I: 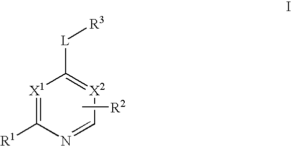
in which:
X¹ and X² are independently selected from the group consisting of —N═ and —CR⁴═, wherein R⁴ is hydrogen or C₁₋₄alkyl;
L is selected from the group consisting of a bond, —O— and —NR⁵—, wherein R⁵ is hydrogen or C₁₋₄alkyl;
R¹ is selected from the group consisting of —X³ NR⁶R⁷, —X³OR⁷ and —X³R⁷, wherein X³ is a bond or C₁₋₄alkylene, R⁶ is hydrogen or C₁₋₄alkyl and R⁷ is selected from the group consisting of C₆₋₁₀aryl and C₅₋₆heteroaryl; wherein any aryl or heteroaryl is optionally substituted with 1 to 3 radicals independently selected from the group consisting of halo, amino, C₁₋₄alkyl, halo-substituted C₁₋₄alkyl, C₁₋₄alkoxy and halo-substituted C₁₋₄alkoxy;
R² is selected from the group consisting of hydrogen, halo, amino, C₁₋₄alkyl, halo-substituted C₁₋₄alkyl, C₁₋₄alkoxy and halo-substituted C₁₋₄alkoxy;
R³ is selected from the group consisting of C₃₋₈heterocycloalkyl-C₀₋₄alkyl, C₅₋₁₀heteroaryl-C₀₋₄alkyl and C₆₋₁₀aryl-C₀₋₄alkyl; wherein any alkyl group is optionally substituted with 1 to 3 radicals selected from the group consisting of hydroxy, halo and amino; and any aryl, heteroaryl or heterocycloalkyl is optionally substituted with 1 to 3 radicals independently selected from the group consisting of halo, nitro, C₁₋₄alkyl, halo-substituted C₁₋₄alkyl, hydroxy-C₁₋₆alkyl, C₁₋₄alkoxy, halo-substituted C₁₋₄alkoxy, phenyl, C₃₋₈heterocycloalkyl, —X³C(O)NR⁸R⁸, —X³C(O)NR⁸R⁹, —X³C(O)R⁹, —X³S(O)NR⁸R⁸, —X³NR⁸R⁹, —X³NR⁸R⁸, —X³S(O)₂NR⁸R⁸, —X³S(O)₂R⁸, —X³S(O)₂R⁹, —X³SNR⁸R⁸, —X³ONR⁸R⁸, —X³C(O)R⁸, —X³NR⁸C(O)R⁸, —X³NR⁸S(O)₂R⁸, —X³S(O)₂NR⁸R⁹, X³NR⁸S(O)₂R⁹, X³NR⁸C(O)R⁹, —X³NR⁸C(O)NR⁸R⁹, —X³NR⁸C(O)NR⁸R⁸, —X³C(O)OR⁸, ═NOR⁸, —X³NR⁸OR⁸, —X³NR⁸(CH₂)₁₄NR⁸R⁸, —X³C(O)NR⁸(CH₂)₁₄NR⁸R, —X³C(O)NR⁸(CH₂)₁ ⁹, —X³C(O)NR⁸(CH₂)₁₋₄OR⁹, —X³O(CH₂)₁₋₄NR⁸R⁸, —X³C(O)NR⁸(CH₂)₁₋₄OR⁸ and X³NR⁸(CH₂)₁₋₄R⁹; wherein phenyl can be further substituted by a radical selected from —NR⁸R⁸ or —C(O)NR⁸R⁸; X³ is as described above; R⁸ is hydrogen, C₁₋₆alkyl, hydroxy-C₁₋₆alkyl or C₂₋₆alkenyl; and R⁹ is hydroxy, C₆₋₁₀aryl-C₀₋₄alkyl, C₆₋₁₀aryl-C₀₋₄alkyloxy, C₅₋₁₀heteroaryl-C₀₋₄alkyl, C₃₋₈heterocycloalkyl-C₀₋₄alkyl or C₃₋₈cycloalkyl; wherein said aryl, heteroaryl, cycloalkyl, heterocycloalkyl or alkyl of R⁹ is further optionally substituted by up to 2 radicals selected from the group consisting of halo, hydroxy, cyano, amino, nitro, C₁₋₄alkyl, hydroxy-C₁₋₆alkyl, halo-substituted C₁₋₄alkyl, C₁₋₄alkoxy, halo-substituted C₁₋₄alkoxy, halo-alkyl-substituted-phenyl, benzoxy, C₅₋₉heteroaryl, C₃₋₈heterocycloalkyl, —C(O)NR⁸R⁸, —S(O)₂NR⁸R⁸, —NR⁸R⁸, —C(O)R¹⁰ and —NR¹¹R¹¹, wherein R¹⁰ is C₅₋₆heteroaryl and R¹¹ is hydroxy-C₁₋₄alkyl;
and the N-oxide derivatives, prodrug derivatives, protected derivatives, individual isomers and mixture of isomers thereof; and the pharmaceutically acceptable salts and solvates (e.g., hydrates) of such compounds.

In a second aspect, the present invention provides a pharmaceutical composition which contains a compound of Formula I or a N-oxide derivative, individual isomers and mixture of isomers thereof, or a pharmaceutically acceptable salt thereof, in admixture with one or more suitable excipients.

In a third aspect, the present invention provides a method of treating a disease in an animal in which inhibition of kinase activity, particularly Bcr-abl activity, can prevent, inhibit or ameliorate the pathology and/or symptomology of the diseases, which method comprises administering to the animal a therapeutically effective amount of a compound of Formula I or a N-oxide derivative, individual isomers and mixture of isomers thereof, or a pharmaceutically acceptable salt thereof.

In a fourth aspect, the present invention provides the use of a compound of Formula I in the manufacture of a medicament for treating a disease in an animal in which kinase activity, particularly Bcr-abl activity, contributes to the pathology and/or symptomology of the disease.

In a fifth aspect, the present invention provides a method for inhibiting Bcr-abl activity, the method comprising contacting Bcr-abl with a compound that binds to a myristoyl binding pocket of Bcr-abl.

In a sixth aspect, the present invention provides a process for preparing compounds of Formula I and the N-oxide derivatives, prodrug derivatives, protected derivatives, individual isomers and mixture of isomers thereof, and the pharmaceutically acceptable salts thereof.

### DETAILED DESCRIPTION OF THE INVENTION

I. Definitions

Unless defined otherwise, all technical and scientific terms used herein generally have the same meaning as commonly understood by one of ordinary skill in the art to which this invention belongs. Generally, the nomenclature used herein and the laboratory procedures for organic and analytical chemistry are those well known and commonly employed in the art.

“Alkyl” means a straight or branched, saturated, aliphatic radical having the number of carbon atoms indicated. “Lower alkyl” has up to and including 7, preferably up to and including 4 carbons. For example, C₁₋₄alkyl includes methyl, ethyl, propyl, butyl, isopropyl or isobutyl. Alkenyl is as defined for alkyl with the inclusion of at least one double bond. For example, alkenyl includes vinyl, propenyl, isopropenyl, butenyl, isobutenyl or butadienyl. “Halo-substituted-alkyl” is alkyl as defined above where some or all of the hydrogen atoms are substituted with halogen atoms. For example, halo-substituted-alkyl includes trifluoromethyl, fluoromethyl, 1,2,3,4,5-pentafluoro-phenyl, etc. “Hydroxy-alkyl” includes, for example, hydroxymethyl, hydroxymethyl, etc.

“Alkoxy” is as defined for alkyl with the inclusion of an oxygen atom, for example, methoxy, ethoxy, etc. “Halo-substituted-alkoxy” is as defined for alkoxy where some or all of the hydrogen atoms are substituted with halogen atoms. For example, halo-substituted-alkoxy includes trifluoromethoxy, etc.

“Aryl” means a monocyclic or fused bicyclic aromatic ring assembly containing six to ten ring carbon atoms. For example, aryl may be phenyl or naphthyl, preferably phenyl. “Arylene” means a divalent radical derived from an aryl group. “Heteroaryl” is as defined for aryl where one or more of the ring members are a heteroatom. For example heteroaryl includes pyridyl, indolyl, indazolyl, quinoxalinyl, quinolinyl, benzofuranyl, benzopyranyl, benzothiopyranyl, benzo[1,3]dioxole, imidazolyl, benzo-imidazolyl, pyrimidinyl, fuiranyl, oxazolyl, isoxazolyl, triazolyl, tetrazolyl, pyrazolyl, thienyl, etc.

“Cycloalkyl” means a saturated or partially unsaturated, monocyclic, fused bicyclic or bridged polycyclic ring assembly containing the number of ring atoms indicated. For example, C₃₋₁₀cycloalkyl includes cyclopropyl, cyclobutyl, cyclopentyl, cyclohexyl, etc. “Heterocycloalkyl” means cycloalkyl, as defined in this application, provided that one or more of the ring carbons indicated, are replaced by a moiety selected from —O—, —N═, —NR—, —C(O)—, —S—, —S(O)— or —S(O)₂—, wherein R is hydrogen, C₁₋₄alkyl or a nitrogen protecting group. For example, C₃₋₈heterocycloalkyl-C₀₋₄alkyl as used in this application to describe compounds of the invention includes morpholino, morpholino-methyl, morpholino-ethyl, pyrrolidinyl, piperazinyl, piperidinyl, piperidinylone, 1,4-dioxa-8-aza-spiro[4.5]dec-8-yl, etc.

“Halogen” (or halo) preferably represents chloro or fluoro, but may also be bromo or iodo.

Pharmaceutically acceptable salts of the acidic compounds of the present invention are salts formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

Similarly acid addition salts, such as of mineral acids, organic carboxylic and organic sulfonic acids, e.g., hydrochloric acid, methanesulfonic acid, maleic acid, are also possible provided a basic group, such as pyridyl, constitutes part of the structure.

“Treat”, “treating” and “treatment” refer to a method of alleviating or abating a disease and/or its attendant symptoms.

“Inhibition”, “inhibits” and “inhibitor” refer to a compound that prohibits or a method of prohibiting, a specific action or function.

“Therapeutically effective amount” refers to that amount of the compound being administered sufficient to prevent development of or alleviate to some extent one or more of the symptoms of the condition or disorder being treated.

“Composition” as used herein is intended to encompass a product comprising the specified ingredients in the specified amounts, as well as any product, which results, directly or indirectly, from combination of the specified ingredients in the specified amounts. By “pharmaceutically acceptable” it is meant the carrier, diluent or excipient must be compatible with the other ingredients of the formulation and deleterious to the recipient thereof.

“Subject” refers to animals such as mammals, including, but not limited to, primates (e.g., humans), cows, sheep, goats, horses, dogs, cats, rabbits, rats, mice and the like. In certain embodiments, the subject is a human.

“IC₅₀” is the concentration of a compound that results in 50% inhibition of activity of a peptide, protein, enzyme or biological process.

“Myristoyl Binding Pocket” is a region of Bcr-abl at which a myristoyl moiety can bind when the BCR-Abl protein is in an appropriate conformation for myristoyl binding. Myristoyl binding pockets are described in, for example, Hantschel et al., “A Myristoyl/Phosphotyrosine Switch Regulates c-Abl” Cell (2003), Vol. 112, 845-857 and Bhushan et al., “Structural Basis for the Autoinhibition of c-Abl Tyrosine Kinase” Cell (2003), Vol. 112, 859-871.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are all intended to be encompassed within the scope of the present invention.

II. General

The fusion protein Bcr-Abl is a result of a reciprocal translocation that fuses the Abl proto-oncogene with the Bcr gene. Bcr-abl is then capable of transforming B-cells through the increase of mitogenic activity. This increase results in a reduction of sensitivity to apoptosis, as well as altering the adhesion and homing of CML progenitor cells. The present invention provides compounds, compositions and methods for the treatment of kinase related disease, particularly PDGF-R, c-Kit and Bcr-abl kinase related diseases. For example, leukemia and other proliferation disorders related to Bcr-abl, can be treated through the inhibition of wild-type and mutant forms of Bcr-abl.

III. Compounds

A. Preferred Compounds

In some embodiments, with reference to compounds of Formula I, compounds of the invention can be of Formula Ia: 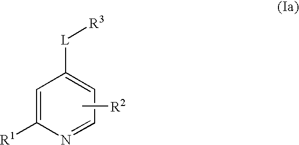
in which L is a bond; R¹ is selected from the group consisting of —NHR⁷, —OR⁷ and —R⁷, wherein R⁷ is phenyl or pyridinyl, optionally substituted with 1 to 3 radicals independently selected from the group consisting of halo, amino, C₁₋₄alkyl, halo-substituted C₁₋₄alkyl, C₁₋₄alkoxy and halo-substituted C₁₋₄alkoxy; and R² is hydrogen or C₁₋₄alkyl.

In a further embodiment, R³ is C₆₋₁₀aryl-C₀₋₄alkyl, optionally substituted with 1 to 3 radicals independently selected from the group consisting of —C(O)NR⁸R⁸, —C(O)NR⁸R⁹, —C(O)R⁹ and —C(O)NR⁸ (CH₂)₂NR⁸R⁸, wherein R⁸ is hydrogen, C₁₋₆alkyl or hydroxy-C₁₋₆alkyl; and R⁹ is C₃₋₈heterocycloalkyl-C₀₋₄alkyl, optionally substituted by —C(O)NR⁸R⁸.

In yet a further embodiment, R¹ is —NHR₇, wherein R⁷ is phenyl substituted with halo-substituted C₄alkyl or halo-substituted C₄alkoxy; R² is hydrogen; and R³ is phenyl substituted with —C(O)NH(CH₂)₂OH, —C(O)NHR⁹, —C(O)R⁹ or —NH(CH₂)₂N(CH₃)₂, wherein R⁹ is morpholino-ethyl or piperidinyl, substituted with —C(O)NH₂.

In another embodiment, compounds of the invention can be of Formula Ib: 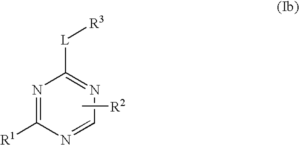
in which L is a bond; R¹ is selected from the group consisting of —NHR⁷, —OR⁷ and —R⁷, wherein R⁷ is phenyl or pyridinyl optionally substituted with 1 to 3 radicals independently selected from the group consisting of halo, amino, C₁₋₄alkyl, halo-substituted C₁₋₄alkyl, C₁₋₄alkoxy and halo-substituted C₁₋₄alkoxy; and R² is hydrogen or C₁₋₄alkyl.

In a further embodiment, R³ is selected from C₅₋₆heteroaryl-C₀₋₄alkyl or C₆₋₁₀aryl-C₀₋₄alkyl; wherein any aryl or heteroaryl is optionally substituted with 1 to 3 radicals selected from the group consisting of C₃₋₈heterocycloalkyl, —C(O)NR⁸R⁸, —C(O)NR⁸R⁹, —C(O)R⁹, —NR⁸R⁹ and —NR⁸(CH₂)₂NR⁸R⁸, wherein R⁸ is hydrogen, C₁₋₆alkyl or hydroxy-C₁₋₆alkyl; and R⁹ is C₆₋₁₀aryl-C₀₋₄alkyl, C₅₋₁₀heteroaryl-C₀₋₄alkyl, C₃₋₈heterocycloalkyl-C₀₋₄alkyl or C₃₋₈cycloalkyl; wherein any aryl, heteroaryl, cycloalkyl, heterocycloalkyl or alkyl of R⁹ is further optionally substituted by up to 2 radicals selected from the group consisting of hydroxy, C₁₋₄alkyl, hydroxy-C₁₋₆alkyl, C₃₋₈heterocycloalkyl, —C(O)NR⁸R³ and —S(O)₂NR⁸R⁸.

In yet a further embodiment, R¹ is —NHR₇, wherein R⁷ is phenyl substituted with halo-substituted C₁₋₄alkyl or halo-substituted C₁₋₄alkoxy; R² is hydrogen; and R³ is pyridinyl or phenyl, optionally substituted with 1 to 3 radicals selected from the group consisting of —C(O)NH(CH₂)₂OH, —C(O)NHCH(C₃H₇)₂CH₂OH, —C(O)NH(CH₂)₂CH₃, —C(O)N(CH₃)₂, —C(O)NH(CH₂)₂N(CH₃)₂, —C(O)NHR⁹, —C(O)N(C₂H₅)R⁹ and —C(O)R⁹, wherein R⁹ is phenyl, phenethyl, pyridinyl, pyrrolidinyl, piperidinyl, morpholino or morpholino-ethyl; wherein any aryl, heteroaryl, heterocycloalkyl or alkyl of R⁹ is further optionally substituted by up to 2 radicals selected from the group consisting of hydroxy, C₁₋₄alkyl, —CH₂OH, —(CH₂)₂OH, pyrrolidinyl, piperazinyl, —C(O)NH₂, —C(O)N(C₂H₅)₂ and —S(O)₂NH₂-

In another embodiment, compounds of the invention can be of Formula Ic: 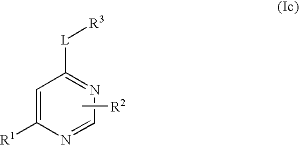
in which L is a bond, —NH—, —N(C₂H₅)— or —O—; R¹ is selected from the group consisting of —NHR⁷, —OR⁷ and —R⁷, wherein R⁷ is phenyl or pyridinyl, optionally substituted with 1 to 3 radicals independently selected from the group consisting of halo, amino, C₁₋₄alkyl, halo-substituted C₁₋₄alkyl, C₁₋₄alkoxy and halo-substituted C₁₋₄alkoxy; and R² is hydrogen or C₁₋₄alkyl.

In a further embodiment, L is a bond; and R³ is selected from the group consisting of C₃₋₈heterocycloalkyl-C₀₋₄alkyl, C₅₋₁₀heteroaryl-C₀₋₄alkyl and C₆₋₁₀aryl-C₀₋₄alkyl; wherein any aryl, heteroaryl or heterocycloalkyl is optionally substituted with 1 to 3 radicals independently selected from the group consisting of halo, nitro, C₁₋₄alkyl, hydroxy-C₁₋₆alkyl, C₁₋₄alkoxy, C₃₋₈heterocycloalkyl, —X³C(O)NR⁸R⁸, —X³C(O)NR⁸R⁹, X³NR⁸R⁹, —X³NR⁸R⁸, —X³S(O)₂NR⁸R⁸, —X³S(O)₂R⁸, —X³S(O)₂R⁹, —X³C(O)R⁸, —X³NR⁸C(O)R⁸, —X³NR⁸S(O)₂R⁸, —X³S(O)₂NR⁸R⁹, —X³NR⁸S(O)₂R⁹, —X³NR⁸C(O)R⁹, —X³NR⁸C(O)NR⁸R⁹, —X³NR⁸C(O)NR⁸R⁸, —X³C(O)OR⁸, ═NOR⁸, —X³NR⁸(CH₂)₁₋₄NR⁸R⁸, —X³C(O)NR⁸(CH₂)₁₋₄NR⁸R⁸ and —X³O(CH₂)₁₋₄NR⁸R⁸; R⁸ is hydrogen, C₁₋₆alkyl or hydroxy-C₁₋₆alkyl; R⁹ is C₆₋₁₀aryl-C₀₋₄alkyl, C₆₋₁₀aryl-C₀₋₄alkyloxy, C₅₋₁₀heteroaryl-C₀₋₄alkyl, C₃₋₈heterocycloalkyl-C₀₋₄alkyl or C₃₋₈cycloalkyl; wherein said aryl, heteroaryl, cycloalkyl, heterocycloalkyl or alkyl of R⁹ is further optionally substituted by up to 2 radicals selected from the group consisting of halo, hydroxy, cyano, nitro, C₁₋₄alkyl, hydroxy-C₁₋₆alkyl, halo-substituted C₁₋₄alkyl, C₁₋₄alkoxy, halo-alkyl-substituted-phenyl, benzoxy, C₅₋₉heteroaryl, C₃₋₈heterocycloalkyl, —C(O)NR⁸R⁸, —S(O)₂NR⁸R⁸, —NR⁸R⁸ and —C(O)R¹⁰, wherein R¹⁰ is C₅₋₆heteroaryl.

In a further embodiment, R³ is selected from the group consisting of morpholino, 1,4-dioxa-8-aza-spiro[4.5]dec-8-yl, 4-oxo-piperidin-1-yl, piperazinyl, pyrrolidinyl, pyridinyl, phenyl, naphthyl, thiophenyl, benzofuran-2-yl, benzo[1,3]dioxolyl, piperidinyl, pyrazinyl, pyrimidinyl, imidazolyl, pyrazolyl and 1H-benzoimidazolyl; wherein any aryl, heteroaryl or heterocycloalkyl is optionally substituted with 1 to 2 radicals independently selected from the group consisting of chloro, methyl, ethyl, hydroxymethyl, methoxy, —C(O)OH, —C(O)H, —C(O)OCH₃, —C(O)N(C₂H₅)₂, —C(O)N(CH₃)₂, —C(O)NHCH₃, —S(O)₂NH₂, —S(O)₂CH₃, chloro, —NH₂, —C(O)CH₃, ═NOCH₃, —NH(CH₂)₂N(CH₃)₂, —NH(CH₂)₃NH₂, —NH(CH₂)₂OH, —C(O)NH(CH₂)₂N(CH₃)₂, —NHR₉, —O(CH₂)₂N(CH₃)₂, morpholino, piperazinyl, —NHC(O)CH₃, —NHC(O)NHC₄H₉, —C(O)NHC₄H₉, —C(O)NHC₃H₇, —C(O)NHC₅H₁₀OH, —C(O)N(C₂H₄OH)₂, —C(O)NHC₂H₄OH, —C(O)NH(CH₂)₂OH, —NHC(O)R⁹, —C(O)NHR⁹, —NHC(O)NHR⁹, —C(O)R⁹, —NHS(O)₂C₄H₉, —NHS(O)₂CH₃, —NHS(O)₂R⁹, —S(O)₂R⁹, —S(O)₂NHR⁹, —C(O)NH₂ and —C(O)NH(CH₂)₂N(CH₃)₂; R⁹ is phenethyl, 2-phenoxy-ethyl, 1H-imidazolyl-propyl, pyridinyl, pyridinyl-methyl, quinolinyl, morpholino, piperidinyl, piperazinyl, pyrrolidinyl, tetrahydro-furan-2-ylmethyl, furan-2-ylmethyl, thiazol-2-ylmethyl, benzo[1,3]dioxol-5-ylmethyl, benzo[1,3]dioxol-5-yl, 3-(2-oxo-pyrrolidin-1-yl)-propyl, 3-imidazol-1-yl-propyl, 3H-pyrazol-3-yl, morpholino-ethyl, phenyl, thiophenyl-methyl, benzyl, cyclohexyl or furan-2-ylmethyl; wherein said aryl, heteroaryl, cycloalkyl, heterocycloalkyl or alkyl of R⁹ is further optionally substituted by up to 2 radicals selected from hydroxy-methyl, hydroxy-ethyl, isobutyl, nitro, amino, hydroxyl, methoxy, trifluoromethoxy, cyano, isopropyl, methyl, ethyl, chloro, fluoro, pyridinyl, morpholino, phenoxy, pyrrolidinyl, trifluoromethyl, trifluoromethyl-substituted-phenyl, —N(CH₃)₂, —C(O)NH₂, —S(O)₂NH₂, —C(O)N(CH₃)₂, cyano or —C(O)R¹⁰; and R¹⁰ is furanyl.

In a further embodiment, L is —NH—, —N(C₂H₅)— or —O—; and R³ is selected from the group consisting of C₅₋₁₀heteroaryl-C₀₋₄alkyl and C₆₋₁₀aryl-C₀₋₄alkyl; wherein any aryl or heteroaryl is optionally substituted with 1 to 3 radicals independently selected from the group consisting of C₁₋₄alkoxy, C₃₋₈heterocycloalkyl, —X³C(O)NR⁸R⁸, —X³S(O)₂NR⁸R⁸, X³NR⁸C(O)R⁸ and —X³NR⁸C(O)NR⁸R⁹; R is hydrogen or C₁₋₆alkyl; and R⁹ is C₆₋₁₀aryl-C₀₋₄alkyl optionally substituted by up to 2 halo-substituted C₁₋₄alkyl radicals.

In yet a further embodiment, R³ is selected from the group consisting of quinolinyl, pyridinyl and phenyl; wherein any aryl or heteroaryl is optionally substituted with 1 to 2 radicals independently selected from the group consisting of morpholino, methoxy, —C(O)NH₂, —NHC(O)NHR⁹ and —S(O)₂NH₂; and R⁹ is phenyl substituted by trifluoromethyl.

Preferred compounds of Formula I are detailed in the Examples and Table I, infra.

B. Preparation of Compounds

The present invention also includes processes for the preparation of compounds of the invention. In the reactions described, it can be necessary to protect reactive functional groups, for example hydroxy, amino, imino, thio or carboxy groups, where these are desired in the final product, to avoid their unwanted participation in the reactions. Conventional protecting groups can be used in accordance with standard practice, for example, see T. W. Greene and P. G. M. Wuts in “Protective Groups in Organic Chemistry”, John Wiley and Sons, 1991.

Compounds of Formula I, wherein L is a bond, can be prepared by proceeding as in the following Reaction Scheme 1: 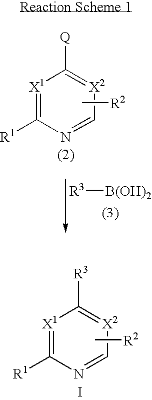
in which X¹, X², R¹, R² and R³ are as defined for Formula I above and Q represents a halo group, for example iodo or chloro, preferably chloro.

Compounds of Formula I can be prepared by reacting a compound of Formula 2 with a compound of Formula 3. The reaction can be effected in the presence of a suitable catalyst (e.g., Pd(PPh₃)₄, etc.), in an appropriate solvent (e.g., acetonitrile) and with an appropriate base (e.g., Na₂CO₃) at 50-100° C. and requires 5-15 hours to complete.

Compounds of Formula I, wherein L is a bond, can also be prepared by proceeding as in the following Reaction Scheme 2: 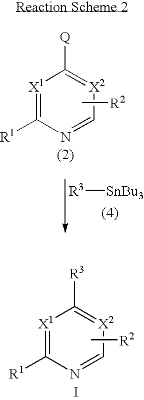
in which X¹, X², R¹, R² and R³ are as defined for Formula I above and Q represents a halo group, for example iodo or chloro, preferably iodo.

Compounds of Formula I can be prepared by reacting a compound of Formula 2 with a compound of Formula 4. The reaction can be effected in the presence of a suitable catalyst (e.g., Pd(PPh₃)₄, etc.) and in an appropriate solvent (e.g., 1,4-dioxane) at 60-110° C. and requires 10-20 hours to complete.

Compounds of Formula I, wherein L is —S, can be prepared by proceeding as in the following Reaction Scheme 3: 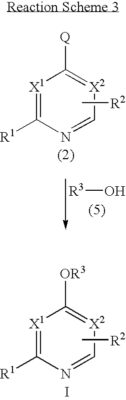
in which X¹, X², R¹, R² and R³ are as defined for Formula I above and Q represents a halo group, for example iodo or chloro, preferably chloro.

Compounds of Formula I can be prepared by reacting a compound of Formula 2 with a compound of Formula 5. The reaction can be effected in the presence of a suitable base (e.g., KO^{t}Bu, etc.) and in an appropriate solvent (e.g., THF) at 50-100° C. and requires 5-10 hours to complete.

Compounds of Formula I, wherein L is —NR⁵—, can be prepared by proceeding as in the following Reaction Scheme 4: 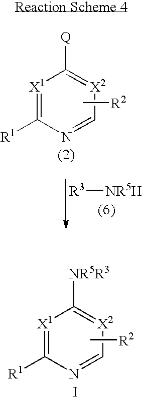
in which X¹, X², R¹, R², R³ and R⁵ are as defined for Formula I above and Q represents a halo group, for example iodo or chloro, preferably chloro.

Compounds of Formula I can be prepared by reacting a compound of Formula 2 with a compound of Formula 6. The reaction can be effected in the presence of a suitable ligand (e.g., IprHCl, etc.), a suitable catalyst (e.g., Pd₂(dba)₃, etc.), a suitable base (e.g., KO^{t}Bu, etc.) and in an appropriate solvent (e.g., 1,4-dioxane, THF, etc.) at 50-100° C. and requires 2-10 hours to complete.

Additional Processes for Preparing Compounds of the Invention

A compound of the invention can be prepared as a pharmaceutically acceptable acid addition salt by reacting the free base form of the compound with a pharmaceutically acceptable inorganic or organic acid. Alternatively, a pharmaceutically acceptable base addition salt of a compound of the invention can be prepared by reacting the free acid form of the compound with a pharmaceutically acceptable inorganic or organic base. Alternatively, the salt forms of the compounds of the invention can be prepared using salts of the starting materials or intermediates.

The free acid or free base forms of the compounds of the invention can be prepared from the corresponding base addition salt or acid addition salt from, respectively. For example a compound of the invention in an acid addition salt form can be converted to the corresponding free base by treating with a suitable base (e.g., ammonium hydroxide solution, sodium hydroxide, and the like). A compound of the invention in a base addition salt form can be converted to the corresponding free acid by treating with a suitable acid (e.g., hydrochloric acid, etc.)

Compounds of the invention in unoxidized form can be prepared from N-oxides of compounds of the invention by treating with a reducing agent (e.g., sulfur, sulfur dioxide, triphenyl phosphine, lithium borohydride, sodium borohydride, phosphorus trichloride, tribromide, or the like) in a suitable inert organic solvent (e.g. acetonitrile, ethanol, aqueous dioxane, or the like) at 0 to 80° C.

Prodrug derivatives of the compounds of the invention can be prepared by methods known to those of ordinary skill in the art (e.g., for further details see Saulnier et al., (1994), Bioorganic and Medicinal Chemistry Letters, Vol. 4, p. 1985). For example, appropriate prodrugs can be prepared by reacting a non-derivatized compound of the invention with a suitable carbamylating agent (e.g., 1,1-acyloxyalkylcarbanochloridate, para-nitrophenyl carbonate, or the like).

Protected derivatives of the compounds of the invention can be made by means known to those of ordinary skill in the art. A detailed description of techniques applicable to the creation of protecting groups and their removal can be found in T. W. Greene, “Protecting Groups in Organic Chemistry”, 3^{rd} edition, John Wiley and Sons, Inc., 1999.

Compounds of the present invention can be conveniently prepared, or formed during the process of the invention, as solvates (e.g., hydrates). Hydrates of compounds of the present invention can be conveniently prepared by recrystallization from an aqueous/organic solvent mixture, using organic solvents such as dioxin, tetrahydrofuran or methanol.

Compounds of the invention can be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers can be carried out using covalent diastereomeric derivatives of the compounds of the invention, dissociable complexes are preferred (e.g., crystalline diastereomeric salts). Diastereomers have distinct physical properties (e.g., melting points, boiling points, solubilities, reactivity, etc.) and can be readily separated by taking advantage of these dissimilarities. The diastereomers can be separated by chromatography, or preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions”, John Wiley And Sons, Inc., 1981.

In summary, the compounds of Formula I can be made by a process, which involves:
(a) reacting a compound of Formula 2 with a compound of Formula 3, 4, 5 or 6: 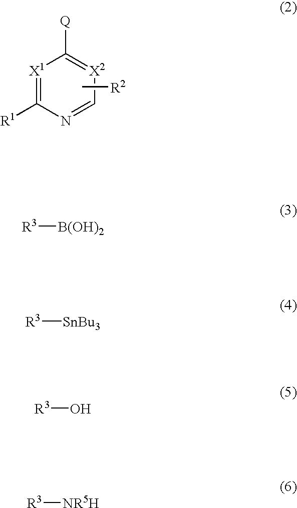
   in which X¹, X², R¹, R², R³ and R⁵ are as defined for Formula I above and Q represents a fluoro, chloro, bromo or iodo; or
(b) optionally converting a compound of the invention into a pharmaceutically acceptable salt;
(c) optionally converting a salt form of a compound of the invention to a non-salt form;
(d) optionally converting an unoxidized form of a compound of the invention into a pharmaceutically acceptable N-oxide;
(e) optionally converting an N-oxide form of a compound of the invention to its unoxidized form;
(f) optionally resolving an individual isomer of a compound of the invention from a mixture of isomers;
(g) optionally converting a non-derivatized compound of the invention into a pharmaceutically acceptable prodrug derivative; and
(h) optionally converting a prodrug derivative of a compound of the invention to its non-derivatized form.

Insofar as the production of the starting materials is not particularly described, the compounds are known or can be prepared analogously to methods known in the art or as disclosed in the Examples hereinafter.

One of skill in the art will appreciate that the above transformations are only representative of methods for preparation of the compounds of the present invention, and that other well known methods can similarly be used.

IV. Compositions

The pharmaceutical compositions according to the invention are those suitable for enteral, such as oral or rectal, transdermal, topical, and parenteral administration to mammals, including man, to inhibit Bcr-abl activity, and for the treatment of Bcr-abl dependent disorders, in particular cancer and tumor diseases, such as leukemias (especially chronic myeloid leukemia and acute lymphoblastic leukemia), and comprise an effective amount of a pharmacologically active compound of the present invention, alone or in combination, with one or more pharmaceutically acceptable carriers.

More particularly, the pharmaceutical compositions comprise an effective Bcr-abl inhibiting amount of a compound of the present invention.

The pharmacologically active compounds of the present invention are useful in the manufacture of pharmaceutical compositions comprising an effective amount thereof in conjunction or mixture with excipients or carriers suitable for either enteral or parenteral application.

Preferred are tablets and gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions are preferably aqueous isotonic solutions or suspensions, and suppositories are preferably prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1 to 75%, preferably about 1 to 50%, of the active ingredient.

Tablets may be either film coated or enteric coated according to methods known in the art.

Suitable formulations for transdermal application include an effective amount of a compound of the present invention with carrier. Preferred carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used.

Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

The pharmaceutical formulations contain an effective Bcr-abl inhibiting amount of a compound of the present invention as defined above, either alone or in combination with another therapeutic agent.

In conjunction with another active ingredient, a compound of the present invention may be administered either simultaneously, before or after the other active ingredient, either separately by the same or different route of administration or together in the same pharmaceutical formulation.

The dosage of active compound administered is dependent on the species of warm-blooded animal (mammal), the body weight, age and individual condition, and on the form of administration. A unit dosage for oral administration to a mammal of about 50 to 70 kg may contain between about 5 and 500 mg of the active ingredient.

V. Methods

The compounds of Formula I in free form or in pharmaceutically acceptable salt form, exhibit valuable pharmacological properties, for example, as indicated by the in, vitro tests described within “Assays”, infra, and are therefore indicated for therapy of diseases and disorders associated with Bcr-abl activity. For Bcr-abl, compounds of Formula I preferably show an IC₅₀ in the range of 1×10⁻¹⁰ to 1×10⁻⁵ M, preferably less than 1 μM for wild-type Bcr-abl and at least two other Bcr-abl mutants (mutants selected from G250E, E255V, T3151, F317L and M351T). For example, compound 97 (Table I) has an IC₅₀ of 0.20, 4.78, 0.25, 5.28, 4.45, and 0.97 for wild-type, G250E, E255V, T3151, F317L and M351T Bcr-abl, respectively.

The invention also provides a method for preventing or treating diseases or conditions comprising abnormal cell growth in a mammal, including a human, comprising administering to the mammal a compound of Formula I in an amount effective to inhibit PDGF-R, c-Kit and/or Bcr-abl activity.

PDGF (Platelet-derived Growth Factor) is a very commonly occurring growth factor, which plays an important role both in normal growth and also in pathological cell proliferation, such as is seen in carcinogenesis and in diseases of the smooth-muscle cells of blood vessels, for example in atherosclerosis and thrombosis.

Compounds of Formula I can inhibit PDGF-R and are, therefore, also suitable for the treatment of tumor diseases, such as gliomas, sarcomas, prostate tumors, and tumors of the colon, breast, and ovary.

The compounds of the present invention also inhibit cellular processes involving stem-cell factor (SCF, also known as the c-kit ligand or steel factor), such as SCF receptor (kit) autophosphorylation and the SCF-stimulated activation of MAPK kinase (mitogen-activated protein kinase).

J The compounds of the present invention, thus inhibit also the autophosphorylation of SCF receptor (and c-kit, a proto-oncogen). MO7e cells are a human promegakaryocytic leukemia cell line, which depends on SCF for proliferation. A compound of Formula I, inhibits the autophosphorylation of SCF—R in the micromolar range.

On the basis of the described properties, the compounds of the present invention, can be used not only as a tumor-inhibiting substance, for example in small cell lung cancer, but also as an agent to treat non-malignant proliferative disorders, such as atherosclerosis, thrombosis, psoriasis, scleroderma, and fibrosis, as well as for the protection of stem cells, for example to combat the hemotoxic effect of chemotherapeutic agents, such as 5-fluoruracil, and in asthma. It can especially be used for the treatment of diseases, which respond to an inhibition of the PDGF-R kinase.

In addition, the compounds of the present invention can be used in combination with other anti-tumor agents.

Also abl kinase, especially v-abl kinase, is inhibited by compounds of the present invention. By analogy, the compounds of the present invention also inhibit Bcr-abl kinase and are thus suitable for the treatment of Bcr-abl-positive cancer and tumor diseases, such as leukemias (especially chronic myeloid leukemia and acute lymphoblastic leukemia, where especially apoptotic mechanisms of action are found), and also shows effects on the subgroup of leukemic stem cells as well as potential for the purification of these cells in vitro after removal of said cells (for example, bone marrow removal) and reimplantation of the cells once they have been cleared of cancer cells (for example, reimplantation of purified bone marrow cells).

In addition, the compounds of the present invention show useful effects in the treatment of disorders arising as a result of transplantation, for example, allogenic transplantation, especially tissue rejection, such as especially obliterative bronchiolitis (OB), i.e. a chronic rejection of allogenic lung transplants. In contrast to patients without OB, those with OB often show an elevated PDGF concentration in bronchoalveolar lavage fluids. Synergistic effects with other immunomodulatory or anti-inflammatory substances are possible, for example when used in combination with cyclosporin, rapamycin, or ascomycin, or immunosuppressant analogues thereof, for example cyclosporin A (CsA), cyclosporin G, FK-506, rapamycin, or comparable compounds, corticosteroids, cyclophosphamide, azathioprine, methotrexate, brequinar, leflunomide, mizoribine, mycophenolic acid, mycophenolate mofetil, 15-deoxyspergualin, immunosuppressant antibodies, especially monoclonal antibodies for leukocyte receptors, for example MHC, CD2, CD3, CD4, CD7, CD25, CD28, B7, CD45, CD58 or their ligands, or other immunomodulatory compounds, such as CTLA41g.

The compounds of the present invention are also effective in diseases associated with vascular smooth-muscle cell migration and proliferation (where PDGF and PDGF-R often also play a role), such as restenosis and atherosclerosis. These effects and the consequences thereof for the proliferation or migration of vascular smooth-muscle cells in vitro and in vivo can be demonstrated by administration of the compounds of the present invention, and also by investigating its effect on the thickening of the vascular intima following mechanical injury in vivo.

Furthermore, the present invention provides a method for inhibiting Bcr-abl activity, the method comprising contacting Bcr-abl with a compound that binds to a myristoyl binding pocket of Bcr-abl. In a preferred embodiment, the compound is a compound of Formula I.

### VI. EXAMPLES

A. Compounds

The present invention is further exemplified, but not limited by, the following examples that illustrate the preparation of compounds of Formula I (Examples), and their intermediates (References), according to the invention.

Reference 1. (6-Chloro-pyrimidin-4-yl)-(4-trifluoromethoxy-phenyl)-amine

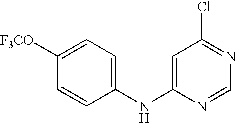

1.0 g 4,6-dichloropyrimidine (6.7 mmol) is dissolved with 1.2g p-trifluoromethoxy aniline (6.7 mmol) in 15 mL ethanol, then 1.75 mL DIEA (10 mmol) is added. Reaction is under reflux for 2 hours, and cooled down to room temperature. After evaporating the solvent, the crude product is purified by flash chromatography (EA/Hexane=3:7) to give (6-Chloro-pyrimidin-4-yl)-(4-trifluoromethoxy-phenyl)-amine as a white solid 1.94 g.

Reference 2. 4-[6-(4-Trifluoromethoxy-phenylamino)-pyrimidin-4-yl]-benzoic Acid

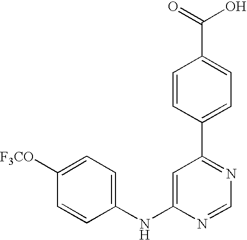

200 mg (4-Chloro-pyrimidin-6-yl)-(4-trifluoromethoxy-phenyl)-amine (0.69 mmol), prepared as in Reference 1, is added to a flask with 115 mg 4-carboxyphenylboronic acid (0.69 mmol), 40 mg palladium tetrakis triphenylphosphine (0.034 mmol) and 292 mg of sodium carbonate (2.76 mmol). Solvent MeCN/H₂O (1:1) 10 mL is added into the flask. After refill with argon, the flask is heated to 90° C. for 8 hours. The hot reaction solution is filtered and collected. 6N HCl solution is added to the solution until the pH is less than 5. The pale solid 4-[6-(4-trifluoromethoxy-phenylamino)-pyrimidin-4-yl]-benzoic acid (220 mg) is collected by filtration and rinsed by 5 mL water twice.

Reference 3. 4-[4-(4-Trifluoromethoxy-phenylamino)-[1,3,5]triazin-2-yl]-benzoic Acid

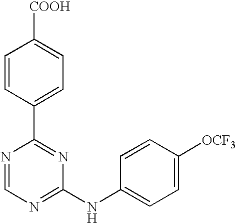

To 100 ml round bottom flask, 1.5 g of 2,4-Dichloro-[1,3,5]triazine (10 mmol), 231 mg of palladium tetrakis triphenylphosphine (0.2 mmol) and 20 ml of 0.5M 4-(ethoxylcarbonyl)-phenyl zinc iodide are mixed. 10 ml of dry THF is added to the reaction mixture. The reaction is carried out at room temperature, overnight. The product is used in the next step without further purification. p-Trifluoromethoxy-aniline (1.77 g; 10 mmol) is added and allowed to react at room temperature for 2 hours. After removal of THF by evaporation, the crude product is redissolved in ethyl acetate (100 ml) and washed with saturated ammonium chloride solution (100 ml; 3 times) and brine (once). The crude product is purified by a silica gel flash column to give 2.8 g of final product as a white solid.

2.8g 4-[4-(4-Trifluoromethoxy-phenylamino)-[1,3,5]triazin-2-yl]-benzoic acid ethyl ester is dissolved in 50 ml of a water/acetonitrile (1:1) mixture. A solution of 19N NaOH (0.74 ml) is added and the reaction is refluxed at 80° C. for 2 hours. The reaction is cooled to room temperature and the pH is adjusted to 5 by the addition of 6N HCl. The light yellow precipitate is collected, washed with 10 ml water and dried to give 4-[4-(4-trifluoromethoxy-phenylamino)-[1,3,5]triazin-2-yl]-benzoic acid (2.4 g). MS: m/z 377.1 (M+H)⁺; ¹H NMR (400 MHz, DMSO) δ 10.62 (s, 1H), 8.92 (s, 1H), 8.51 (d, J=8.0 Hz, 2H), 8.14(d, J=8.1 Hz, 2H), 7.99(d, J=8.1 Hz, 2H), 7.54 (s, 1H), 7.35 (d, J=8.0 Hz, 2H).

### Example 1

### N,N-Dimethyl-4-[6-(4-trifluoromethoxy-phenylamino)-pyrimidin-4-yl]-benzamide

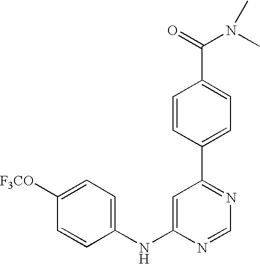

100 mg 4-[6-(4-trifluoromethoxy-phenylamino)-pyrimidin-4-yl]-benzoic acid (0.27 mmol), prepared as in Reference 2, is added to 200 μL dimethylamine (2.0 M in THF, 0.40 mmol), HATU (112 mg; 0.30 mmol) and DEA (232 μL; 1.33 mmol). After adding 4 mL solvent DMF, the reaction is stirred at room temperature for 8 hours. The solvent is removed and the crude product is purified by flash chromatography using MeOH/DCM (5%/95%) resulting in N,N-dimethyl-4-[6-(4-trifluoromethoxy-phenylamino)-pyrimidin-4-yl]-benzamide as a pale yellow solid (101 mg). MS: m/z 402.1 (M+H)⁺; ¹H NMR (400 MHz, DMSO) δ 8.80 (s, 1H), 8.05 (d, J=8.1 Hz, 2H), 7.83 (d, J=9.1 Hz, 2H), 7.58 (d, J=8.4 Hz, 2H), 7.37 (d, J=8.4 Hz, 2H), 7.30 (s, 1H), 2.97 (s, 6H).

### Example 2

### N-(2-Morpholin-4-yl-ethyl)-4-[6-(4-trifluoromethoxy-phenylamino)-pyrimidin-4-yl]-benzamide

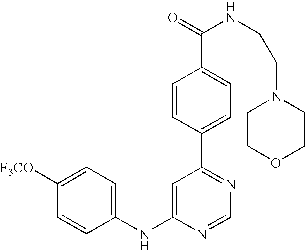

100 mg 4-[6-(4-trifluoromethoxy-phenylamino)-pyrimidin-4-yl]-benzoic acid (0.27 mmol), prepared as in Reference 2, is added to 4-(2-aminoethyl)morpholine (53 μL; 0.40 mmol), HATU (112 mg; 0.30 mmol) and DIEA (232 μL; 1.33 mmol). DMF (4 mL) is added and the reaction stirred at room temperature for 8 hours. The solvent is removed and the crude product is purified by flash chromatography using MeOH/DCM (5%:95%) resulting in N-(2-morpholin-4-yl-ethyl)-4-[6-(4-trifluoromethoxy-phenylamino)-pyrimidin-4-yl]-benzamide as a pale yellow solid (123 mg). MS: m/z 488.1 (M+H)⁺; ¹H NMR (400 MHz, DMSO) δ 8.78 (s, 1H), 8.16 (d, J=8.3 Hz, 2H), 8.03 (d, J=8.5 Hz, 2H), 7.85 (d, J=10.2 Hz, 2H), 7.36 (d, J=8.8 Hz, 2H), 7.34 (s, 1H), 4.01 (t, 7.0 Hz, 2H), 3.66 (t, 6.8 Hz, 4H), 3.57 (t, 7.2 Hz, 2H), 3.35 (t, 6.9 Hz, 4H).

### Example 3

### (6-Pyridin-4-yl-pyrimidin-4-yl)-(4-trifluoromethoxy-phenyl)-amine

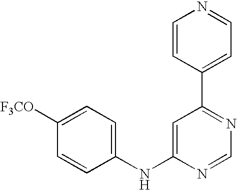

(4-Chloro-pyrimidin-6-yl)-(4-trifluoromethoxy-phenyl)-armine (100 mg; 0.35 mmol), prepared as in Reference 1, is added to 4-(tributyltin)-pyridine (190 mg; 0.52 mmol) and palladium tetrakis triphenylphosphine (20 mg; 0.018 mmol). Solvent is dry 1,4-dioxane. The reaction is carried out at reflux temperatures, under argon gas, for 16 hours. After removing the solvent, the crude product is purified by flash chromatography using Hexane/EA (35%:65%) resulting in (6-Pyridin-4-yl-pyrimidin-4-yl)-(4-trifluoromethoxy-phenyl)-amine as a yellow solid (40 mg). MS: m/z 333.2 (M+H)⁺; ¹H NMR (400 MHz, CDCl₃) δ 8.83 (s, 1H), 8.79 (d, J=8.2 Hz, 2H), 7.82 (d, J=9.0 Hz, 2H), 7.51 (d, J=8.4 Hz, 2H), 7.29 (d, J=8.4 Hz, 2H), 7.09 (s, 1H).

### Example 4

### [6-(1,4-Dioxa-8-aza-spiro[4.5]dec-8-yl)-pyrimidin-4-yl]-(4-trifluoromethoxy-phenyl)-amine

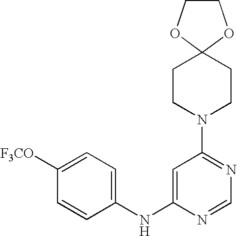

(4-Chloro-pyrimidin-6-yl)-(4-trifluoromethoxy-phenyl)-amine (100 mg; 0.35 mmol), prepared as in Reference 1, is added to 1,4-dioxa-8-aza-spiro-[4.5]-decane (75 mg; 0.52 mmol), tris-(dibenzylidene-acetone)-dipalladium (0) (8.1 mg; 0.009 mmol), 1,3-bis(2,6-di-1-propylphenyl)-imidazolium chloride 7.4 mg (0.018 mmol) and potassium tert-butoxide (59 mg; 0.52 mmol). Solvent is dry 1,4-dioxane. The reaction is carried out at 80° C. for 4 hours under argon gas. After removing the solvent, the crude product is purified by flash chromatography using Hexane/EA (40%/60%) resulting in [6-(1,4-dioxa-8-aza-spiro[4.5]dec-8-yl)-pyrimidin-4-yl]-(4-trifluoromethoxy-phenyl)-amine as a white solid (110 mg). MS: m/z 397.2 (M+H)⁺; ¹H NMR (400 MHz, CDCl₃) δ 8.27 (s, 1H), 7.33 (d, J=8.2 Hz, 2H), 7.18 (d, J=8.4 Hz, 2H), 6.66 (s, 1H), 3.99 (t, J=4.8 Hz, 4H), 3.67 (t, J=5.2 Hz, 4H), 1.70 (t, J=5.5 Hz, 4H).

### Example 5

### [6-(3-Methanesulfonyl-phenyl)-pyrimidin-4-yl]-(4-trifluoromethoxy-phenyl)-amine

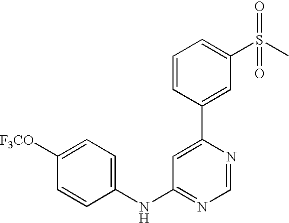

To a degassed solution of (6-chloropyrimidin-4-yl)-(4-trifluoromethoxyphenyl)-amine (510 mg, 1.76 mmol), prepared as in Reference 1, and (3-methylsulfonylphenyl)-boronic acid (352 mg, 1.76 mmol) in 0.4 M sodium carbonate aqueous solution (17 mL) and acetonitrile (17 mL) is added PPh₃ (100 mg, 0.09 mmol). After stirring at about 90° C. under N₂ for 12 hours, the reaction mixture is partitioned between saturated NaHCO₃ and CHCl₃. The aqueous layer is extracted with additional CHCl₃. The combined organic layers are dried over MgSO₄, filtered and concentrated under reduced pressure. The resultant yellowish oil is purified by column chromatography (SiO₂, hexane/ethyl acetate (4/6)) to give [6-(3-methane-sulfonylphenyl)-pyrimidin-4-yl]-(4-trifluoromethoxyphenyl)-amine as a pale yellowish solid (619 mg; 86%). ¹H NMR (400 MHz, CDCl₃) δ 8.81 (s, 1H), 8.55-8.54 (m, 1H), 8.30-8.28 (m, 1H), 8.10-8.03 (m, 1H), 7.71-7.68 (m, 1H), 7.55-7.53 (m, 2H), 7.28-7.27 (m, 1H), 7.10-7.09 (m, 2H), 3.11 (s, 3H).

### Example 6

### 3-[6-(4-Trifluoromethoxy-phenylamino)-pyrimidin-4-yl]-benzamide

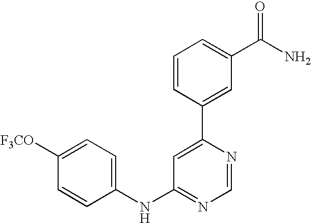

To a degassed solution of (6-chloropyrimidin-4-yl)-(4-trifluoromethoxyphenyl)-amine (73 mg, 0.25 mmol), prepared as in Reference 1, and (3-aminocarbonylphenyl)-boronic acid (42 mg, 0.25 mmol) in 0.4 M sodium carbonate aqueous solution (1.3 mL) and acetonitrile (1.3 mL) was added PPh₃ (15 mg, 0.01 mmol). After stirring at about 90° C. under N₂ for 12 hours, the reaction mixture is partitioned between saturated NaHCO₃ and CHCl₃/2-propanol (4/1). The aqueous layer is extracted with additional CHCl₃/2-propanol (4/1) and the combined organic layers are dried over MgSO₄, filtered, and concentrated under reduced pressure. The resultant yellowish oil is purified by column chromatography (SiO₂, ethyl acetate) to give 3-[6-(4-trifluoromethoxyphenyl-amino)-pyrimidin-4-yl]-benzamide as a white solid (82 mg; 88%). MSm/z375.10(M+1).]

### Example 7

### [6-(3-Amino-phenyl)-pyrimidin-4-yl]-(4-trifluoromethoxy-phenyl)-amine

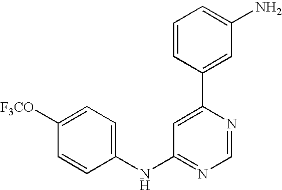

To a degassed solution of (6-chloropyrimidin-4-yl)-(4-trifluoromethoxyphenyl)-amine (217 mg, 0.75 mmol), prepared as in Reference 1, and (2-aminophenyl)-boronic acid (130 mg, 0.75 mmol) in 0.4 M sodium carbonate aqueous solution (3.8 mL) and acetonitrile (3.8 mL) is added PPh₃ (45 mg, 0.04 mmol). The reaction mixture is stirred at about 90° C. under N₂ for 12 hours and the hot suspension is filtered. The filtrate is concentrated under reduced pressure to give a crude product, which is purified by column chromatography (SiO₂, hexane/ethyl acetate (4/1)) to give [6-(3-aminophenyl)-pyrimidin-4-yl]-(4-trifluoro-methoxyphenyl)-amine as a pale yellowish solid (218 mg; 84%). MS m/z 347.10 (M+1).

### Example 8

### N-(2-Hydroxy-ethyl)-4-[4-(4-trifluoromethoxy-phenylamino)-[1,3,5]triazin-2-yl]-benzamide

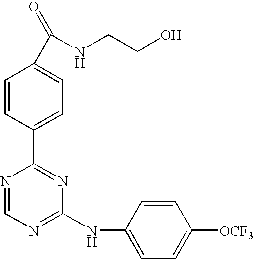

4-[4-(4-Trifluoromethoxy-phenylamino)-[1,3,5]triazin-2-yl]-benzoic acid (50 mg; 0.13 mmol), prepared as in Reference 3, is mixed with ethanol-amine (12 μl; 0.2 mmol), HATU (54 mg, 1.5 mmol) in dry DMF (0.5 ml) and DIEA (113 μl; 0.65 mmol). The reaction is carried out at room temperature, overnight. After removing solvent, the final product is purified by reverse phase HPLC, 5-95% acetonitrile in 10 minutes to give N-(2-hydroxy ethyl)-4-[4-(4-trifluoromethoxy-phenylamino)-[1,3,5]triazin-2-yl]-benzamide. MS: m/z 420.1 (M+H)⁺; ¹H NMR (400 MHz, DMSO) δ 10.52 (s, 1H), 8.84 (s, 1H), 8.55 (t, J=6.0 Hz, 1H), 8.40(d, J=8.1 Hz, 2H), 7.98(d, J=9.5 Hz, 2H), 7.86 (s, 2H), 7.36 (d, J=8.0 Hz, 2H), 3.62 (s, 1H), 3.47(t, J=6 Hz, 2H), 3.31(dd, J=5.9, 2H).

### Example 9

### N-(2-Dimethylamino-ethyl)-4-[4-(4-trifluoromethoxy-phenylamino)-[1,3,5]triazin-2-yl]-benzamide

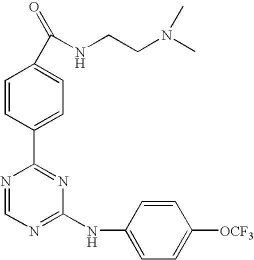

4-[4-(4-Trifluoromethoxy-phenylamino)-[1,3,5]triazin-2-yl]-benzoic acid (50 mg, 0.13 mmol), prepared as in Reference 3, is mixed with N,N-dimethyl-ethane-1,2-diamine (22 μl; 0.2 mmol), HATU (54 mg; 1.5 mmol) in 0.5 ml dry DMF and DIEA (113 μl, 0.65 mmol). The reaction is carried out at room temperature, overnight. After removing solvent, the final product is purified by reverse phase HPLC, 5-95% acetonitrile in 10 minutes, to give N-(2-Dimethylamino-ethyl)-4-[4-(4-trifluoromethoxy-phenylamino)-[1,3,5]triazin-2-yl]-benzamide. MS: m/z 447.2 (M+H)⁺; ¹H NMR (400 MHz, DMSO) δ 10.52 (s, 1H), 9.32(S, 1 h), 8.84 (s, 1H), 8.79 (t, J=4.5 Hz, 1H), 8.42(d, J=8.1 Hz, 2H), 7.98(d, J=8.2 Hz, 2H), 7.86 (s, 2H), 7.35 (d, J=8.0 Hz, 2H), 3.58 (dd, J=5.8 Hz, 2H), 3.24(dd, J=5.9, 2H), 2.81(d, J=4.8).

By repeating the procedures described in the above examples, using appropriate starting materials, the following compounds of Formula I, as identified in Examples 10-14 and Table 1, are obtained.

### Example 10

### N-(2-Morpholin-4-yl-ethyl)-N′-(4-trifluoromethoxy-phenyl)-pyrimidine-4,6-diamine

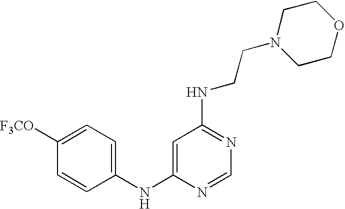

White solid. MS: m/z 384.2 (M+H)⁺, ¹H NMR (400 MHz, CDCl₃) δ 8.21 (s, 1H), 7.76 (s, 1H), 7.34 (d, J=8.2 Hz, 2H), 7.20 (d, J=8.4 Hz, 2H), 5.89 (s, 1H), 3.69 (t, J=4.7 Hz, 4H), 2.27(d, J=4.3 Hz, 2H), 2.58 (t, J=5.2 Hz, 2H), 2.45 (t, J=5.3 Hz, 4H).

### Example 11

### (6-Imidazol-1-yl-pyrimidin-4-yl)-(4-trifluoromethoxy-phenyl)-amine

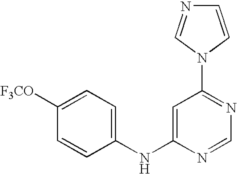

White solid. MS: m/z 322.1 (M+H)⁺, ¹H NMR (400 MHz, DMSO) δ 9.15 (s, 1H), 8.67 (s, 1H), 8.12 (s, 1H), 7.77 (d, J=7.2 Hz, 2H), 7.51 (s, 1H), 7.40 (d, J=8.2 Hz, 2H), 7.05 (s, 1H).

### Example 12

### {6-[2-(3-Imidazol-1-yl-propylamino)-pyridin-4-yl]-pyrimidin-4-yl}-(4-trifluoromethoxy-phenyl)-amine

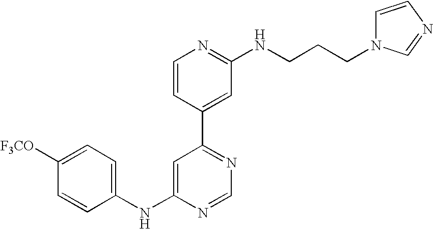

Yellow solid. MS: m/z 456.2 (M+H)⁺, ¹H NMR (400 MHz, DMSO) δ 9.13 (s, 1H), 8.78 (s, 1H), 8.12 (d, J=6.1 Hz, 1H), 7.84 (d, J=7.2 Hz, 2H), 7.81 (s, 1H), 7.71 (s, 1H), 7.43 (s, 1H), 7.37 (d, J=8.5 Hz, 2H), 7.32 (s, 1H), 7.16 (d, J=5.9 Hz, 1H), 4.30 (t, d=6.7 Hz, 2H), 3.36 (t, J=6.8 Hz, 2H), 2.16 (m, 2H).

### Example 13

### 3-[6-(4-Trifluoromethoxy-phenylamino)-pyrimidin-4-yl]-benzenesulfonamide

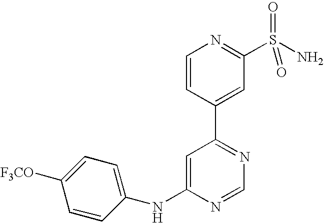

Pale yellow solid. MS: m/z 411.1 (M+H)⁺; ¹H NMR (400 MHz, DMSO) δ 8.79 (s, 1H), 8.53 (s, 1H), 8.23 (d, J=8.5 HZ, 1H), 7.96 (d, J=5.1 Hz, 1H), 7.85 (d, J=6.9 Hz, 2H), 7.75 (t, J=7.9 Hz, 1H), 7.48 (s, 2H), 7.36 (d, J=8.2 Hz, 2H), 7.33 (s, 1H).

### Example 14

### N-(2-Hydroxy-ethyl)-4-{4-[6-(4-trifluoromethoxy-phenylamino)-pyrimidin-4-yl]-pyridin-2-yl}-benzamide

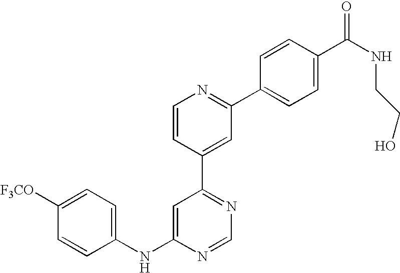

Pale yellow solid. MS: m/z 496.2 (M+H)⁺; ¹H NMR (400 MHz, DMSO) δ 8.88 (d, J=5.1 Hz, 1H), 8.85 (s, 1H), 8.55 (s, 2H), 8.25 (d, J=8.4 Hz, 2H), 8.02(d, 8.5 Hz, 2H), 7.96 (dd, J=5.2 Hz, 1H), 7.87 (d, J=8.7 Hz, 2H), 7.58(m, 2H), 7.49 (s, 1H), 7.38 (d, J=8.5 Hz, 2H), 3.54 (t, J=6.1 Hz, 2H), 3.37 (m, 2H).

TABLE 1

| Compound | Structure | MS (m/z) |
|---|---|---|
| 15 | 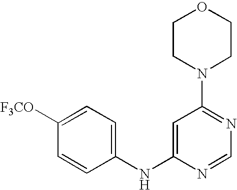 | 341.1 |
| 16 | 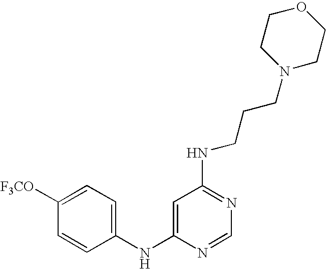 | 398.2 |
| 17 | 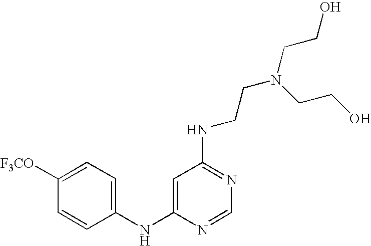 | 402.2 |
| 18 | 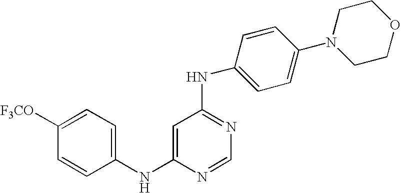 | 432.2 |
| 19 | 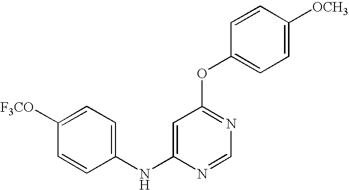 | 378.1 |
| 20 | 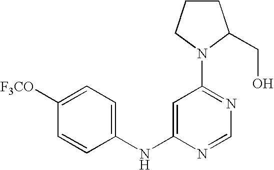 | 355.1 |
| 21 | 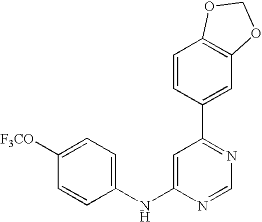 | 376.1 |
| 22 | 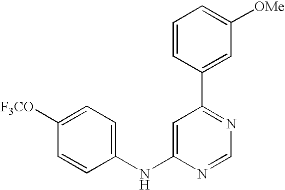 | 362.1 |
| 23 | 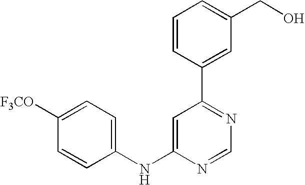 | 362.1 |
| 24 | 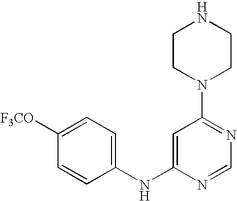 | 340.1 |
| 25 | 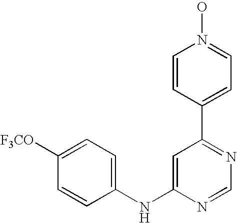 | 349.1 |
| 26 | 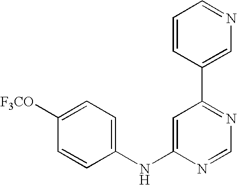 | 333.1 |
| 27 | 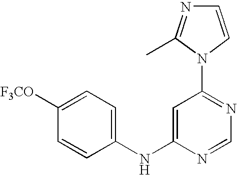 | 336.1 |
| 28 | 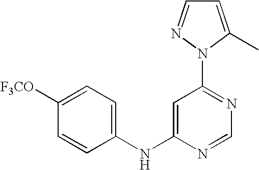 | 336.1 |
| 29 | 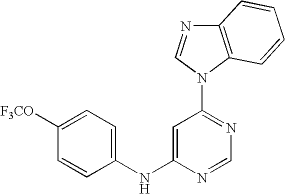 | 372.1 |
| 30 | 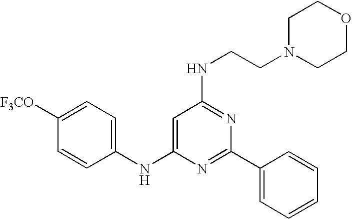 | 460.2 |
| 31 | 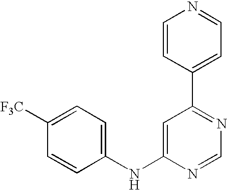 | 317.1 |
| 32 | 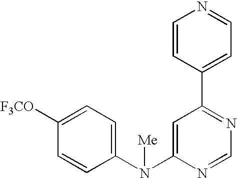 | 347.1 |
| 33 | 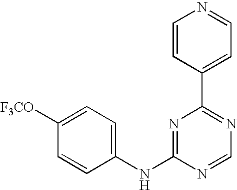 | 334.1 |
| 34 | 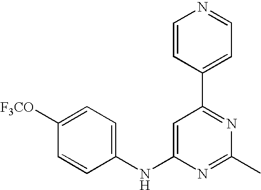 | 347.1 |
| 35 | 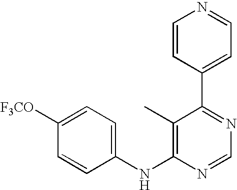 | 347.1 |
| 36 | 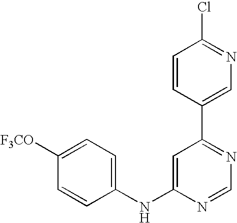 | 367.1 |
| 37 | 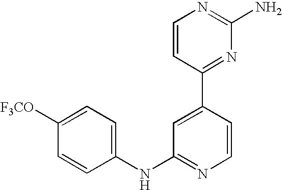 | 348.1 |
| 38 | 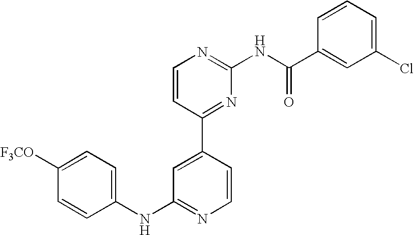 | 486.1 |
| 39 | 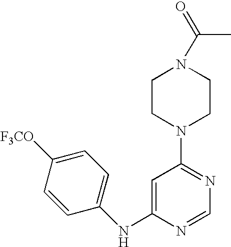 | 382.2 |
| 40 | 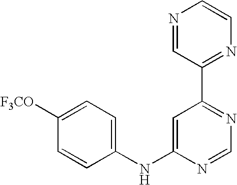 | 334.1 |
| 41 | 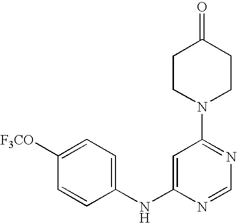 | 353.1 |
| 42 | 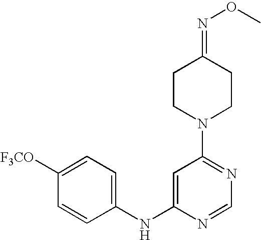 | 382.2 |
| 43 | 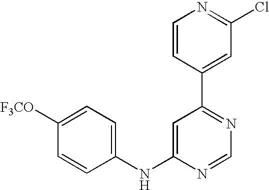 | 366.05 |
| 44 | 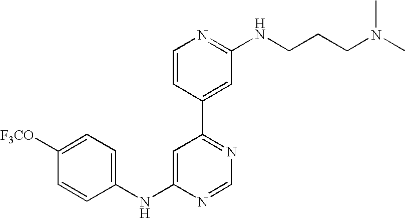 | 433.2 |
| 45 | 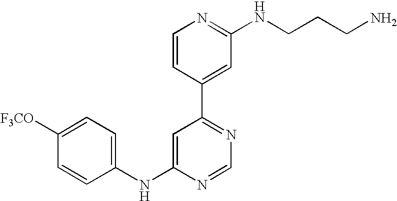 | 404.16 |
| 46 | 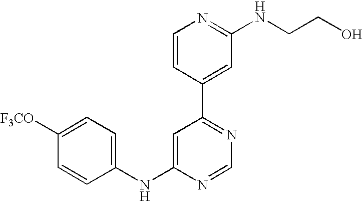 | 392.1 |
| 47 | 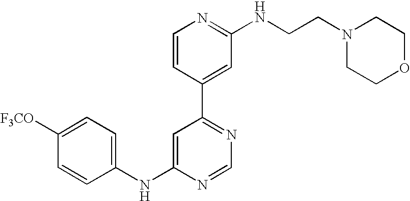 | 461.2 |
| 48 | 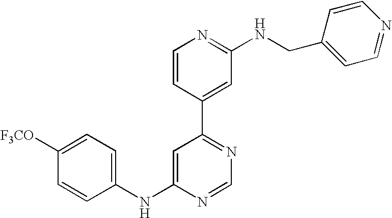 | 438.14 |
| 49 | 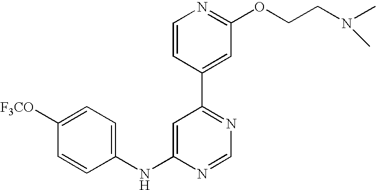 | 420.2 |
| 50 | 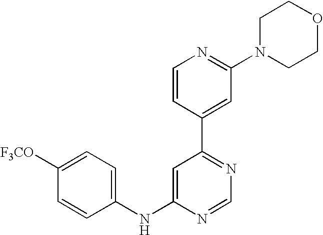 | 418.2 |
| 51 | 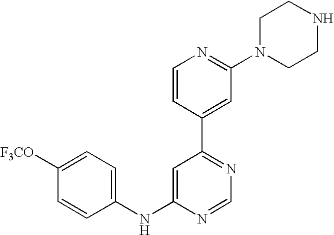 | 416.16 |
| 52 | 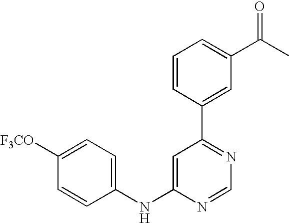 | 374.1 |
| 53 | 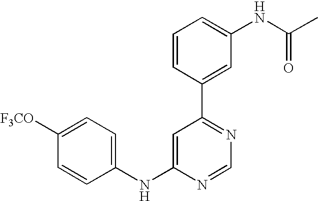 | 389.1 |
| 54 | 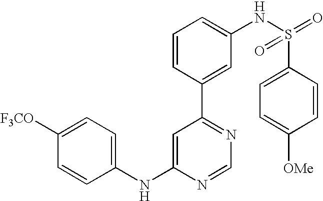 | 517.1 |
| 55 | 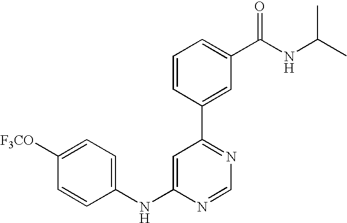 | 417.2 |
| 56 | 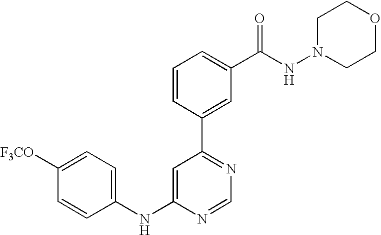 | 459.15 |
| 56 | 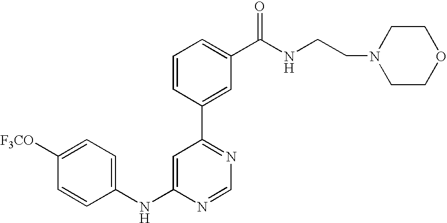 | 488.2 |
| 57 | 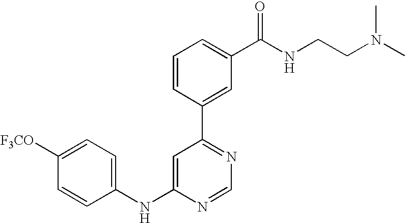 | 446.2 |
| 58 | 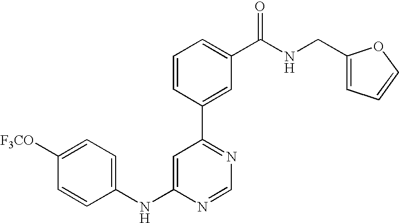 | 455.2 |
| 59 | 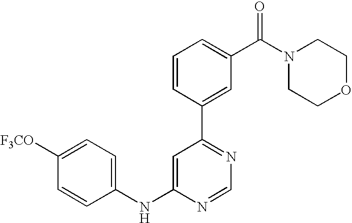 | 445.2 |
| 60 | 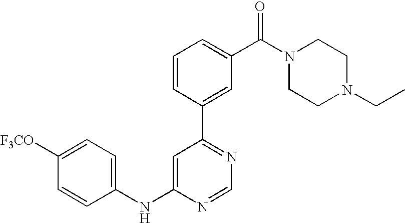 | 472.2 |
| 61 | 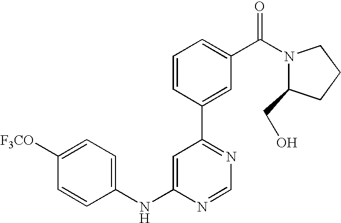 | 459.2 |
| 62 | 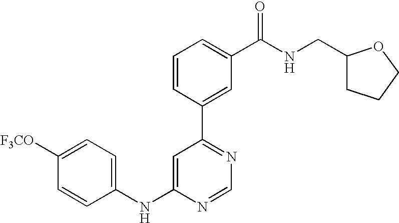 | 459.2 |
| 63 | 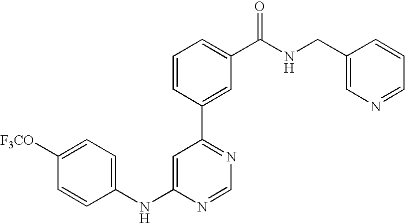 | 465.14 |
| 64 | 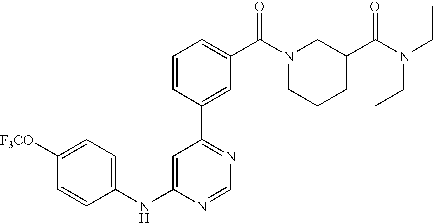 | 541.23 |
| 65 | 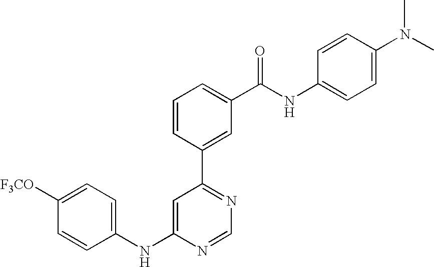 | 494.2 |
| 66 | 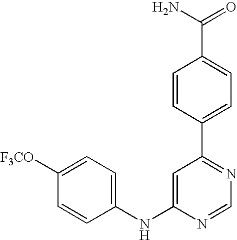 | 375.2 |
| 67 | 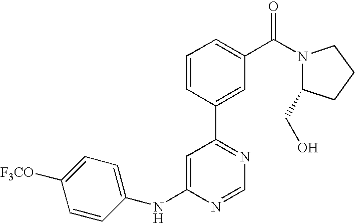 | 458.16 |
| 68 | 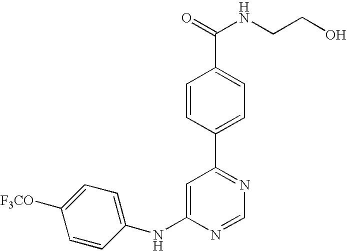 | 419.2 |
| 69 | 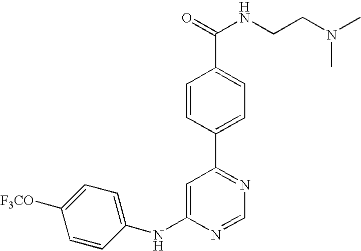 | 445.17 |
| 70 | 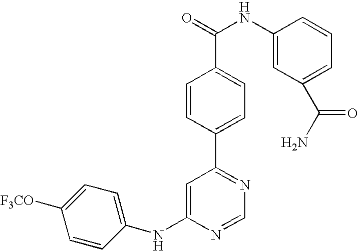 | 494.2 |
| 71 | 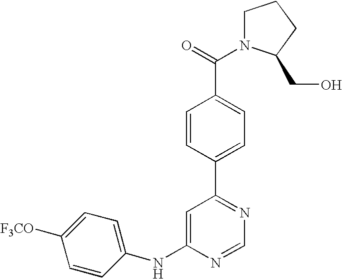 | 459.2 |
| 72 | 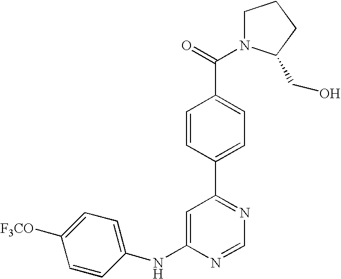 | 458.16 |
| 73 | 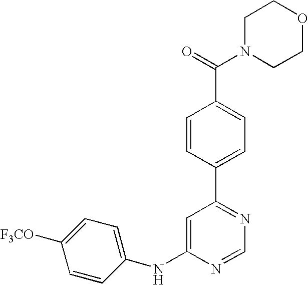 | 445.2 |
| 74 | 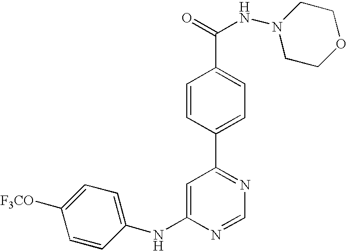 | 459.15 |
| 75 | 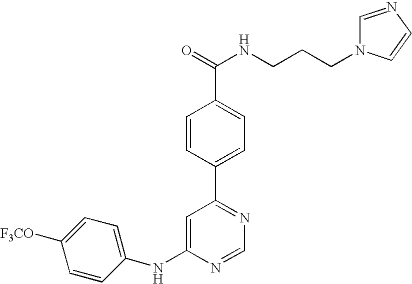 | 482.17 |
| 76 | 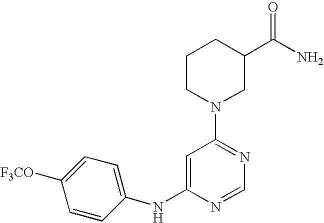 | 382.2 |
| 77 | 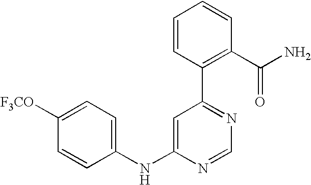 | 375.2 |
| 78 | 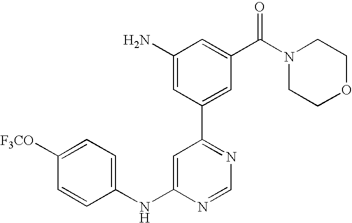 | 460.2 |
| 79 | 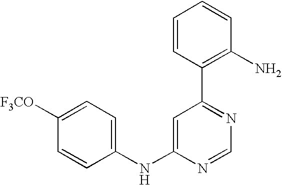 | 346.2 |
| 80 | 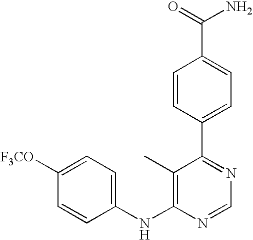 | 389.2 |
| 81 | 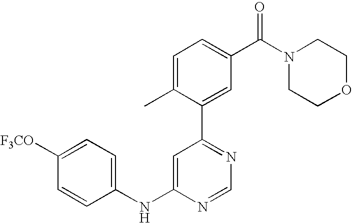 | 459.2 |
| 82 | 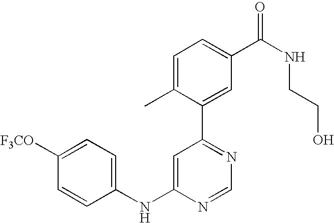 | 432.14 |
| 83 | 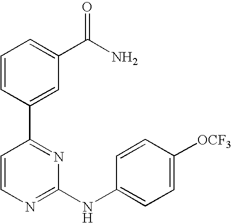 | 375.2 |
| 84 | 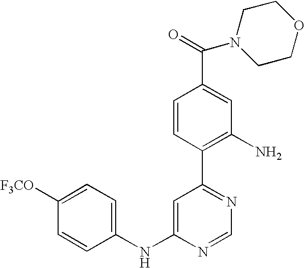 | 460.2 |
| 85 | 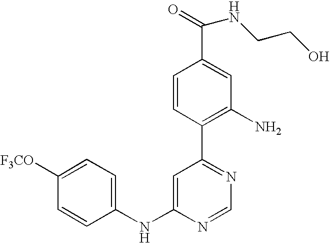 | 433.14 |
| 86 | 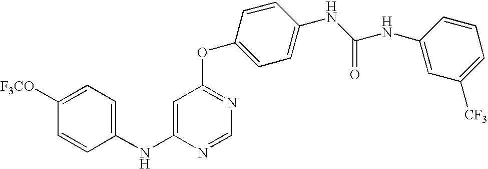 | 550.2 |
| 87 | 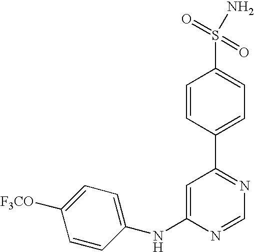 | 411.1 |
| 88 | 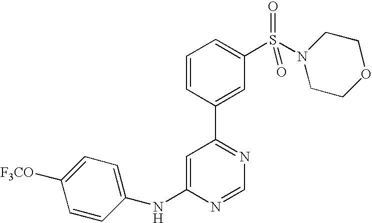 | 481.2 |
| 89 | 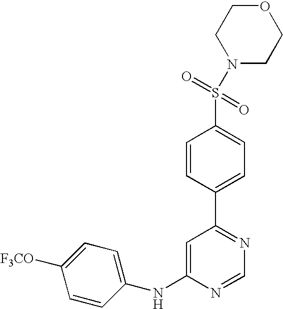 | 481.2 |
| 90 | 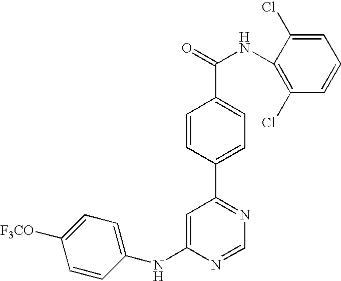 | 518.05 |
| 91 | 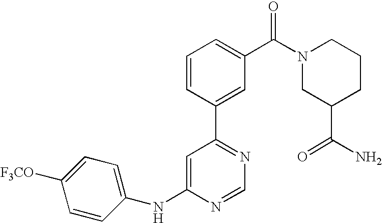 | 485.17 |
| 92 | 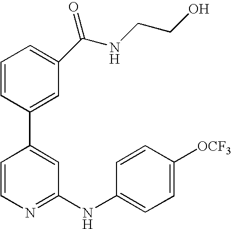 | 418.2 |
| 93 | 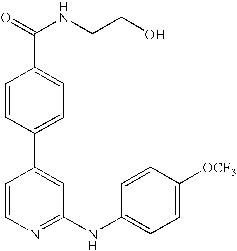 | 418.2 |
| 94 | 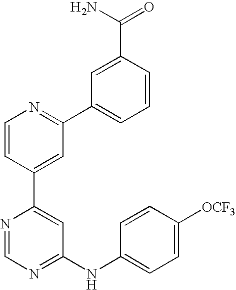 | 452.1 |
| 95 | 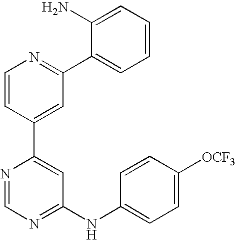 | 424.2 |
| 96 | 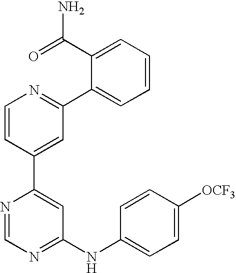 | 452.2 |
| 97 | 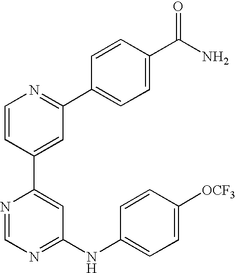 | 452.2 |
| 98 | 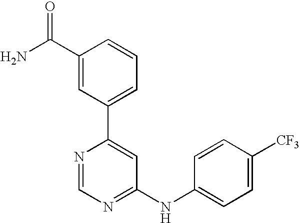 | 358.10 |
| 99 | 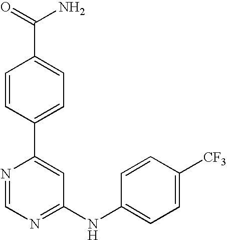 | 359.2 |
| 100 | 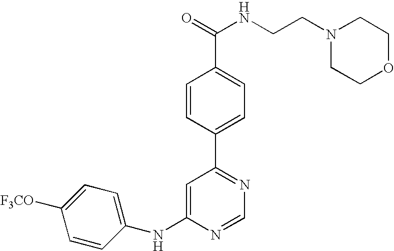 | 472.2 |
| 101 | 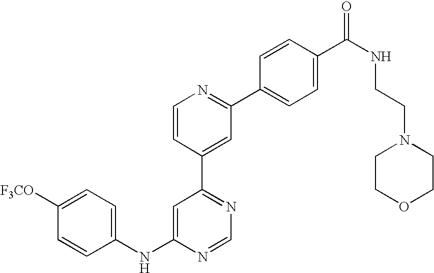 | 565.2 |
| 102 | 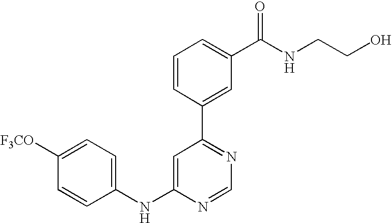 | 418.13 |
| 103 | 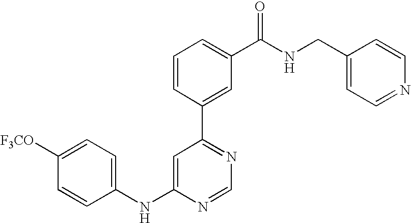 | 465.14 |
| 104 | 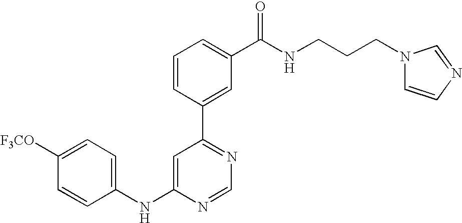 | 483.2 |
| 105 | 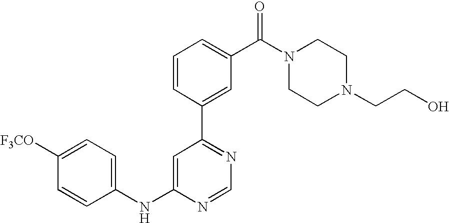 | 488.2 |
| 106 | 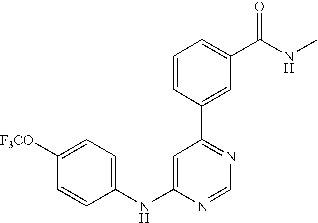 | 388.11 |
| 107 | 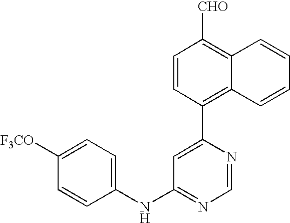 | 410.1 |
| 108 | 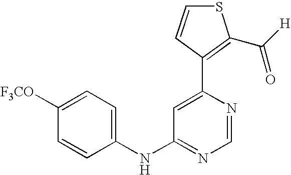 | 366.1 |
| 109 | 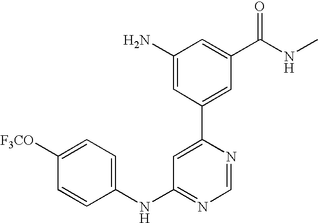 | 404.1 |
| 110 | 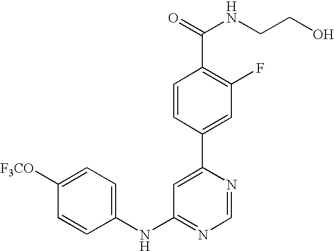 | 437.1 |
| 111 | 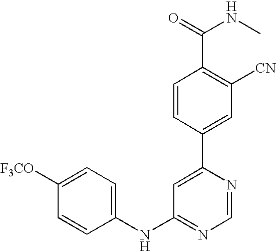 | 414.1 |
| 112 | 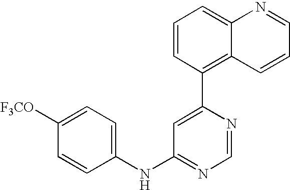 | 382.10 |
| 113 | 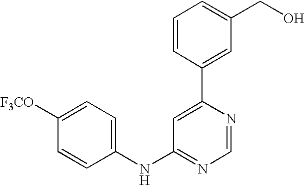 | 361.10 |
| 114 | 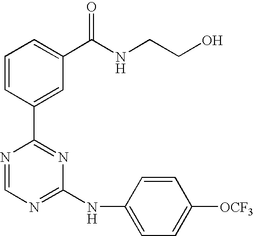 | 420.2 |
| 115 | 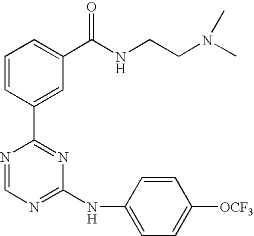 | 446.17 |
| 116 | 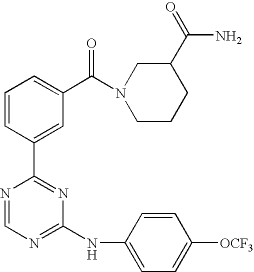 | 487.2 |
| 117 | 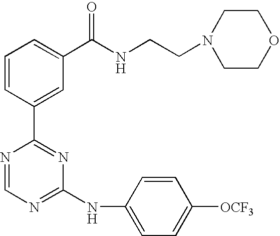 | 489.2 |
| 118 | 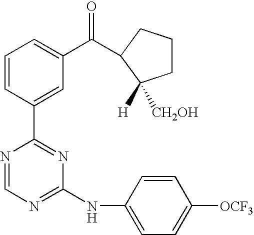 | 459.15 |
| 119 | 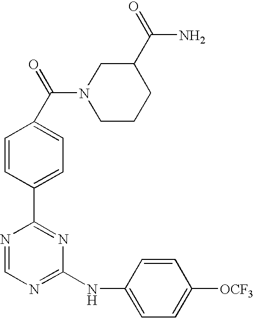 | 486.16 |
| 120 | 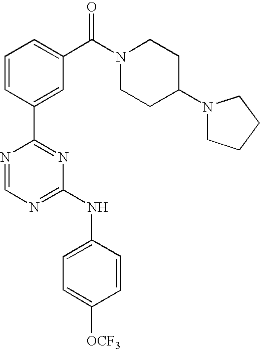 | 512.21 |
| 121 | 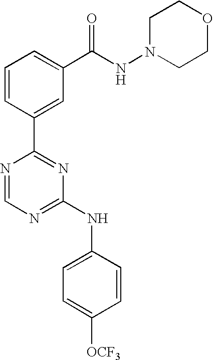 | 460.15 |
| 122 | 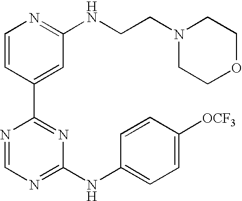 | 462.2 |
| 123 | 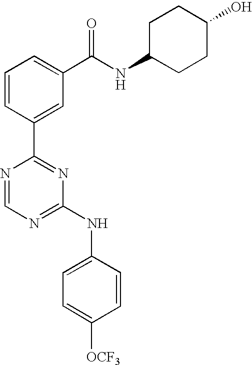 | 473.17 |
| 124 | 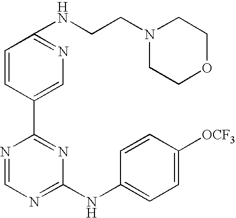 | 462.2 |
| 125 | 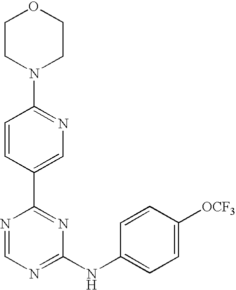 | 419.2 |
| 126 | 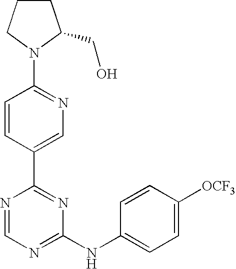 | 432.15 |

B. Assays

Compounds of the present invention are assayed to measure their capacity to selectively inhibit cell proliferation of 32D cells expressing Bcr-abl (32D-p210) compared with parental 32D cells. Compounds selectively inhibiting the proliferation of these Bcr-abl transformed cells are tested for anti-proliferative activity on Ba/F3 cells expressing either wild type or the mutant forms of Bcr-abl.

Inhibition of Cellular Bcr-abl Dependent Proliferation (High Throughput Method)

The murine cell line used is the 32D hemopoietic progenitor cell line transformed with Bcr-abl cDNA (32D-p210). These cells are maintained in RPMI/10% fetal calf serum (RPMI/FCS) supplemented with penicillin 50 μg/mL, streptomycin 50 μg/mL and L-glutamine 200 mM. Untransformed 32D cells are similarly maintained with the addition of 15% of WEHI conditioned medium as a source of IL3.

50 μl of a 32D or 32D-p210 cells suspension are plated in Greiner 384 well microplates (black) at a density of 5000 cells per well. 50nl of test compound (1 mM in DMSO stock solution) is added to each well (ST1571 is included as a positive control). The cells are incubated for 72 hours at 37° C., 5% CO₂. 10 μl of a 60% Alamar Blue solution (Tek diagnostics) is added to each well and the cells are incubated for an additional 24 hours. The fluorescence intensity (Excitation at 530 nm, Emission at 580 nm) is quantified using the Acquestm system (Molecular Devices).

Inhibition of Cellular Bcr-Abl Dependent Proliferation

32D-p210 cells are plated into 96 well TC plates at a density of 15,000 cells per well. 50 μL of two fold serial dilutions of the test compound (Cₘₐₓ is 40 μM) are added to each well (ST1571 is included as a positive control). After incubating the cells for 48 hours at 37° C., 5% CO₂, 15 μL of MTT (Promega) is added to each well and the cells are incubated for an additional 5 hours. The optical density at 570 nm is quantified spectrophotometrically and IC₅₀ values, the concentration of compound required for 50% inhibition, determined from a dose response curve.

Effect on Cell Cycle Distribution

32D and 32D-p210 cells are plated into 6 well TC plates at 2.5×10⁶ cells per well in 5 ml of medium and test compound at 1 or 10 μM is added (STI571 is included as a control). The cells are then incubated for 24 or 48 hours at 37° C., 5% CO₂. 2 ml of cell suspension is washed with PBS, fixed in 70% EtOH for 1 hour and treated with PBS/EDTA/RNase A for 30 minutes. Propidium iodide (Cf=10 μg/ml) is added and the fluorescence intensity is quantified by flow cytometry on the FACScalibur™ system (BD Biosciences). Test compounds of the present invention demonstrate an apoptotic effect on the 32D-p210 cells but do not induce apoptosis in the 32D parental cells.

Effect on Cellular Bcr-abl Autophosphorylation

Bcr-abl autophosphorylation is quantified with capture Elisa using a c-abl specific capture antibody and an antiphosphotyrosine antibody. 32D-p210 cells are plated in 96 well TC plates at 2×10⁵ cells per well in 50 μL of medium. 50 μL of two fold serial dilutions of test compounds (Cₘₐₓ is 10 μM) are added to each well (STI571 is included as a positive control). The cells are incubated for 90 minutes at 37° C., 5% CO₂. The cells are then treated for 1 hour on ice with 150 μL of lysis buffer (50 mM Tris-HCl, pH 7.4, 150 mM NaCl, 5 mM EDTA, 1 mM EGTA and 1% NP-40) containing protease and phosphatase inhibitors. 50 μL of cell lysate is added to 96 well optiplates previously coated with anti-abl specific antibody and blocked. The plates are incubated for 4 hours at 4° C. After washing with TBS-Tween 20 buffer, 50 μL of alkaline-phosphatase conjugated anti-phosphotyrosine antibody is added and the plate is further incubated overnight at 4° C. After washing with TBS-Tween 20 buffer, 90 μL of a luminescent substrate are added and the luminescence is quantified using the Acquest™ system (Molecular Devices). Test compounds of the invention that inhibit the proliferation of the Bcr-abl expressing cells, inhibit the cellular Bcr-abl autophosphorylation in a dose-dependent manner.

### Effect on Proliferation of Cells Expressing Mutant Forms of Bcr-Abl

Compounds of the invention are tested for their antiproliferative effect on Ba/F3 cells expressing either wild type or the mutant forms of Bcr-abl (G250E, E255V, T315I, F317L, M351T) that confers resistance or diminished sensitivity to STI571. The antiproliferative effect of these compounds on the mutant-Bcr-abl expressing cells and on the non transformed cells were tested at 10, 3.3, 1.1 and 0.37 μM as described above (in media lacking IL3). The IC₅₀ values of the compounds lacking toxicity on the untransformed cells were determined from the dose response curves obtained as describe above.

Although the foregoing invention has been described in some detail by way of illustration and example for purposes of clarity of understanding, it will be obvious that certain changes and modifications may be practiced within the scope of the appended claims. In addition, each Reference provided herein is incorporated by Reference in its entirety to the same extent as if each Reference was individually incorporated by Reference.

1. A compound of Formula I: 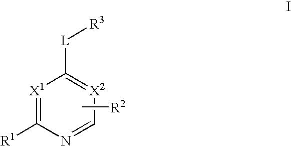
in which:
X¹ and X² are independently selected from the group consisting of —N=and —CR⁴═, wherein R⁴ is hydrogen or C₁₋₄alkyl;
L is selected from the group consisting of a bond, —O— and —NW—, wherein R⁵ is hydrogen or C₁₋₄alkyl;
R¹ is selected from the group consisting of —X³NR⁶R⁷, —X³OR⁷ and —X³R⁷, wherein X³ is a bond or C₁₋₄alkylene, R⁶ is hydrogen or C₁₋₄alkyl and R⁷ is selected from the group consisting of C₆₋₁₀aryl and C₅₋₆heteroaryl; wherein any aryl or heteroaryl is optionally substituted with 1 to 3 radicals independently selected from the group consisting of halo, amino, C₁₋₄alkyl, halo-substituted C₁₋₄alkyl, C₁₋₄alkoxy and halo-substituted C₄alkoxy;
R² is selected from the group consisting of hydrogen, halo, amino, C₁₋₄alkyl, halo-substituted C₁₋₄alkyl, C₁₋₄alkoxy and halo-substituted C₁₋₄alkoxy;
R³ is selected from the group consisting of C₃₋₈heterocycloalkyl-C₀₋₄alkyl, C₅₋₁₀heteroaryl-C₀₋₄alkyl, C₆₋₁₀aryl-C₀₋₄alkyl and —X³NR⁸R⁸; wherein any alkyl group is optionally substituted with 1 to 3 radicals selected from the group consisting of hydroxy, halo and amino; and any aryl, heteroaryl or heterocycloalkyl is optionally substituted with 1 to 3 radicals independently selected from the group consisting of halo, nitro, C₁₋₄alkyl, halo-substituted C₁₋₄alkyl, hydroxy-C₁₋₆alkyl, C₁₋₄alkoxy, halo-substituted C₁₋₄alkoxy, phenyl, C₃₋₈heterocycloalkyl, —X³C(O)NR⁸R⁸, —X³C(O)NR⁸R⁹, —X³C(O)R⁹, —X³S(O)NR⁸R⁸, —X³NR⁸R⁹, —X³NR⁸R⁸, —X³S(O)₂NR⁸R⁸, —X³S(O)₂R⁸, —X³S(O)₂R⁹, _X³SNR⁸R⁸, —X³ONR⁸R⁸, —X³C(O)R⁸, —X³NR⁸C(O)R⁸, —X³NR⁸S(O)₂R⁸, —X³S(O)₂NR⁸R⁹, X³NR⁸S(O)₂R⁹, —X³NR⁸C(O)R⁹, —X³NR⁸C(O)NR⁸R⁹, —X³NR⁸C(O)NR⁸R⁸, —X³C(O)OR⁸, ═NOR⁸, —X³NR⁸OR⁸, —X³NR⁸(CH₂)₁₋₄NR⁸R⁸, —X³C(O)NR⁸(CH₂)₁₋₄NR⁸R⁸, —X³C(O)NR⁸(CH₂)₁₄R⁹, —X³C(O)NR⁸(CH₂)₁₋₄OR⁹, —X³O(CH₂)₁₋₄NR⁸R⁸, —X³C(O)NR⁸(CH₂)₁₋₄OR⁸ and X³NR⁸(CH₂)₁₋₄R⁹; wherein phenyl can be further substituted by a radical selected from —NR⁸R⁸ or —C(O)NR⁸R⁸; X³ is as described above; R⁸ is hydrogen, C₁₋₆alkyl, hydroxy-C₁₋₆alkyl or C₂₋₆alkenyl; and R⁹ is hydroxy, C₆₋₁₀aryl-C₀₋₄alkyl, C₆₋₁₀aryl-C₀₋₄alkyloxy, C₅₀heteroaryl-C₀₋₄alkyl, C₃₋₈heterocycloalkyl-C₀₋₄alkyl or C₃₋₈cycloalkyl; wherein said aryl, heteroaryl, cycloalkyl, heterocycloalkyl or alkyl of R⁹ is further optionally substituted by up to 2 radicals selected from the group consisting of halo, hydroxy, cyano, amino, nitro, C₁₋₄alkyl, hydroxy-C₁₋₆alkyl, halo-substituted C₁₋₄alkyl, C₁₋₄alkoxy, halo-substituted C₁₋₄alkoxy, halo-alkyl-substituted-phenyl, benzoxy, C₅₋₉heteroaryl, C₃₋₈heterocycloalkyl, —C(O)NR⁸R⁸, —S(O)₂NR⁸R⁸, —NR⁸R⁸, —C(O)R¹⁰ and —NR¹¹R¹¹, wherein R¹⁰ is C₅₋₆heteroaryl and R¹¹ is hydroxy-C₁₋₄alkyl; and
the pharmaceutically acceptable salts, hydrates, solvates, isomers and prodrugs thereof.

2. The compounds of claim 1 of Formula Ia: 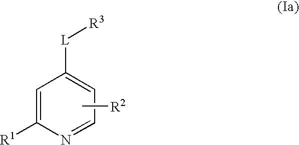
in which
L is a bond;
R¹ is selected from the group consisting of —NHR⁷, —OR⁷ and —R⁷, wherein R⁷ is phenyl or pyridinyl, optionally substituted with 1 to 3 radicals independently selected from the group consisting of halo, amino, C₁₋₄alkyl, halo-substituted C₁₋₄alkyl, C₁₋₄alkoxy and halo-substituted C₁₋₄alkoxy;
R² is hydrogen or C₁₋₄alkyl; and
R³ is C₆₋₁₀aryl-C₀₋₄alkyl, optionally substituted with 1 to 3 radicals independently selected from the group consisting of —C(O)NR R⁸, —C(O)NR⁸R⁹, —C(O)R⁹ and —C(O)NR⁸(CH₂)₂NR⁸R⁸, wherein R⁸ is hydrogen, C₁₋₆alkyl or hydroxy-C₁₋₆alkyl; and R⁹ is C₃₋₈heterocycloalkyl-C₀₋₄alkyl, optionally substituted by —C(O)NR R³.

3. The compounds of claim 2 in which
R¹ is —NHR⁷, wherein R⁷ is phenyl substituted with halo-substituted C₁₋₄alkyl or halo-substituted C₁₋₄alkoxy;
R² is hydrogen; and
R³ is phenyl substituted with —C(O)NH(CH₂)₂OH, —C(O)NHR⁹, —C(O)R⁹ or —NH(CH₂)₂N(CH₃)₂, wherein R⁹ is morpholino-ethyl or piperidinyl, substituted with —C(O)NH₂.

4. The compounds of claim 1 of Formula Ib: 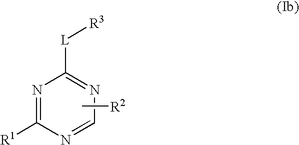
in which
L is a bond;
R¹ is selected from the group consisting of —NHR⁷, —OR⁷ and —R⁷, wherein R⁷ is phenyl or pyridinyl optionally substituted with 1 to 3 radicals independently selected from the group consisting of halo, amino, C₁₋₄alkyl, halo-substituted C₁₋₄alkyl, C₁₋₄alkoxy and halo-substituted C₁₋₄alkoxy;
R² is hydrogen or C₁₋₄alkyl; and
R³ is selected from C₅₋₆heteroaryl-C₀₋₄alkyl or C₆₋₁₀aryl-C₀₋₄alkyl; wherein any aryl or heteroaryl is optionally substituted with 1 to 3 radicals selected from the group consisting of C₃₋₈heterocycloalkyl, —C(O)NR⁸R⁸, —C(O)NR⁵R⁹, —C(O)R⁹, —NR⁸R⁹ and —NR⁸(CH₂)₂NR⁸R⁸, wherein R⁸ is hydrogen, C₁₋₆alkyl or hydroxy-C₁₋₆alkyl; and R⁹ is C₆₋₁₀aryl-C₀₋₄alkyl, C₅₋₁₀heteroaryl-C₀₋₄alkyl, C₃₋₈heterocycloalkyl-C₀₋₄alkyl or C₃₋₈cycloalkyl; wherein any aryl, heteroaryl, cycloalkyl, heterocycloalkyl or alkyl of R⁹ is further optionally substituted by up to 2 radicals selected from the group consisting of hydroxy, C₁₋₄alkyl, hydroxy-C₁₋₆alkyl, C₃₋₈heterocycloalkyl, —C(O)NR⁸R⁸ and —S(O)₂NR⁸R⁸.

5. The compounds of claim 4 in which
R¹ is —NHR⁷, wherein R⁷ is phenyl substituted with halo-substituted C₁₋₄alkyl or halo-substituted C₁₋₄alkoxy;
R² is hydrogen; and
R³ is pyridinyl or phenyl, optionally substituted with 1 to 3 radicals selected from the group consisting of —C(O)NH(CH₂)₂OH, —C(O)NHCH(C₃H₇)₂CH₂OH, —C(O)NH(CH₂)₂CH₃, —C(O)N(CH₃)₂, —C(O)NH(CH₂)₂N(CH₃)₂, —C(O)NHR⁹, —C(O)N(C₂H₅)R⁹ and —C(O)R⁹, wherein R⁹ is phenyl, phenethyl, pyridinyl, pyrrolidinyl, piperidinyl, morpholino or morpholino-ethyl; wherein any aryl, heteroaryl, heterocycloalkyl or alkyl of R⁹ is further optionally substituted by up to 2 radicals selected from the group consisting of hydroxy, C₁₋₄alkyl, —CH₂OH, —(CH₂)₂OH, pyrrolidinyl, piperazinyl, —C(O)NH₂, —C(O)N(C₂H₅)₂ and —S(O)₂NH₂.

6. The compounds of claim 1 of Formula Ic: 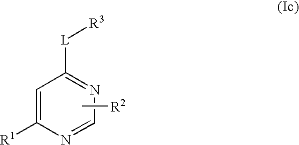
in which
L is a bond, —NH—, —N(C₂H₅)— or —O—;
R¹ is selected from the group consisting of —NHR⁷, —OR⁷ and —R⁷, wherein R⁷ is phenyl or pyridinyl, optionally substituted with 1 to 3 radicals independently selected from the group consisting of halo, amino, C₁₋₄alkyl, halo-substituted C₁₋₄alkyl, C₁₋₄alkoxy and halo-substituted C₁₋₄alkoxy; and
R² is hydrogen or C₁₋₄alkyl.

7. The compounds of claim 6 in which
L is a bond; and
R³ is selected from the group consisting of C₃₋₈heterocycloalkyl-C₀₋₄alkyl, C₅₋₁₀heteroaryl-C₀₋₄alkyl and C₆₋₁₀aryl-C₀₋₄alkyl; wherein any aryl, heteroaryl or heterocycloalkyl is optionally substituted with 1 to 3 radicals independently selected from the group consisting of halo, nitro, C₁₋₄alkyl, hydroxy-C₁₋₆alkyl, C₁₋₄alkoxy, C₃₋₈heterocycloalkyl, —X³C(O)NR⁸R⁸, —X³C(O)NR⁸R⁹, —X³NR⁸R⁹, X³NR⁸R⁸, —X³S(O)₂NR⁸R⁸, —X³S(O)₂R⁸, —X³S(O)₂R⁹, —X³C(O)R⁸, —X³NR⁸C(O)R⁸, —X³NR⁸S(O)₂R⁸, —X³S(O)₂NR⁸R⁹, —X³NR⁸S(O)₂R⁹, —X³NR⁸C(O)R⁹, —X³NR⁸C(O)NR⁸R⁹, —X³NR⁸C(O)NR⁸R⁸, —X³C(O)OR⁸, ═NOR⁸, —X³NR⁸ (CH₂)18 R⁸, —X³C(O)NR⁸(CH₂)₁₋₄NR⁸R⁸ and —X³O(CH₂)₁₋₄NR⁸R⁸; R⁸ is hydrogen, C₁₋₆alkyl or hydroxy-C₁₋₆alkyl; R⁹ is C₆₋₁₀aryl-C₀₋₄alkyl, C₆₋₁₀aryl-C₀₋₄alkyloxy, C₅₋₁₀heteroaryl-C₀₋₄alkyl, C₃₋₈heterocycloalkyl-C₀₋₄alkyl or C₃₋₈cycloalkyl; wherein said aryl, heteroaryl, cycloalkyl, heterocycloalkyl or alkyl of R⁹ is further optionally substituted by up to 2 radicals selected from the group consisting of halo, hydroxy, cyano, nitro, C₁₋₄alkyl, hydroxy-C₁₋₆alkyl, halo-substituted C₁₋₄alkyl, C₁₋₄alkoxy, halo-alkyl-substituted-phenyl, benzoxy, C₅₋₁₀heteroaryl, C₃₋₈heterocycloalkyl, —C(O)NR⁸R⁸, S(O)₂NR⁸R⁸, —NR⁸R⁸ and —C(O)R¹⁰, wherein R¹⁰ is C₅₋₆heteroaryl.

8. The compounds of claim 7 in which R³ is selected from the group consisting of morpholino, 1,4-dioxa-8-aza-spiro[4.5]dec-8-yl, 4-oxo-piperidin-1-yl, piperazinyl, pyrrolidinyl, pyridinyl, phenyl, naphthyl, thiophenyl, benzofuran-2-yl, benzo[1,3]dioxolyl, piperidinyl, pyrazinyl, pyrimidinyl, imidazolyl, pyrazolyl and 1H-benzoimidazolyl; wherein any aryl, heteroaryl or heterocycloalkyl is optionally substituted with 1 to 2 radicals independently selected from the group consisting of chloro, methyl, ethyl, hydroxymethyl, methoxy, —C(O)OH, —C(O)H, —C(O)OCH₃, —C(O)N(C₂H₅)₂, —C(O)N(CH₃)₂, —C(O)NHCH₃, —S(O)₂NH₂, —S(O)₂CH₃, chloro, —NH₂, —C(O)CH₃, ═NOCH₃, —NH(CH₂)₂N(CH₃)₂, —NH(CH₂)₃NH₂, —NH(CH₂)₂OH, —C(O)NH(CH₂)₂N(CH₃)₂, —NHR⁹, —O(CH₂)₂N(CH₃)₂, morpholino, piperazinyl, —NHC(O)CH₃, —NHC(O)NHC₄H₉, —C(O)NHC₄H₉, —C(O)NHC₃H₇, —C(O)NHC₅H₁₀OH, —C(O)N(C₂H₄OH)₂, —C(O)NHC₂H₄OH, —C(O)NH(CH₂)₂OH, —NHC(O)R⁹, —C(O)NHR⁹, —NHC(O)NHR⁹, —C(O)R⁹, —NHS(O)₂C₄H₉, —NHS(O)₂CH₃, —NHS(O)₂R⁹, —S(O)₂R⁹, —S(O)₂NHR⁹, —C(O)NH₂ and —C(O)NH(CH₂)₂N(CH₃)₂; R⁹ is phenethyl, 2-phenoxy-ethyl, 1H-imidazolyl-propyl, pyridinyl, pyridinyl-methyl, quinolinyl, morpholino, piperidinyl, piperazinyl, pyrrolidinyl, tetrahydro-furan-2-ylmethyl, furan-2-ylmethyl, thiazol-2-ylmethyl, benzo[1,3]dioxol-5-ylmethyl, benzo[1,3]dioxol-5-yl, 3-(2-oxo-pyrrolidin-1-yl)-propyl, 3-imidazol-1-yl-propyl, 3H-pyrazol-3-yl, morpholino-ethyl, phenyl, thiophenyl-methyl, benzyl, cyclohexyl or furan-2-ylmethyl; wherein said aryl, heteroaryl, cycloalkyl, heterocycloalkyl or alkyl of R⁹ is further optionally substituted by up to 2 radicals selected from hydroxy-methyl, hydroxy-ethyl, isobutyl, nitro, amino, hydroxyl, methoxy, trifluoromethoxy, cyano, isopropyl, methyl, ethyl, chloro, fluoro, pyridinyl, morpholino, phenoxy, pyrrolidinyl, trifluoromethyl, trifluoromethyl-substituted-phenyl, —N(CH₃)₂, —C(O)NH₂, —S(O)₂NH₂, —C(O)N(CH₃)₂, cyano or —C(O)R¹⁰; and R¹⁰ is furanyl.

9. The compounds of claim 6 in which
L is —NH—, —N(C₂H₅)— or —O—; and
R³ is selected from the group consisting of C₅₋₁₀heteroaryl-C₀₋₄alkyl and C₆₋₁₀aryl-C₀₋₄alkyl; wherein any aryl or heteroaryl is optionally substituted with 1 to 3 radicals independently selected from the group consisting of C₁₋₄alkoxy, C₃₋₈heterocycloalkyl, —X³C(O)NR⁸R⁸, X³S(O)₂NR⁸R⁸, —X³NR⁸C(O)R⁸ and —X³NR⁸C(O)NR⁸R⁹; R⁸ is hydrogen or C₁₋₆alkyl; and R⁹ is C₆₋₁₀aryl-C₀₋₄alkyl optionally substituted by up to 2 halo-substituted C₁₋₄alkyl radicals.

10. The compounds of claim 9 in which R³ is selected from the group consisting of quinolinyl, pyridinyl and phenyl; wherein any aryl or heteroaryl is optionally substituted with 1 to 2 radicals independently selected from the group consisting of morpholino, methoxy, —C(O)NH₂, —NHC(O)NHR⁹ and —S(O)₂NH₂; and R⁹ is phenyl substituted by trifluoromethyl.

11. A pharmaceutical composition for the treatment of tumors in warm-blooded animals, comprising an effective amount of a compound of claim 1.

12. A method of treatment of warm-blooded animals suffering from a tumoral disease, comprising treating warm-blooded animals in need of such treatment with an effective tumor-inhibiting amount of a compound of claim 1.

13. The method of claim 12, wherein said tumor disease is responsive to inhibition of a tyrosine protein kinase.

14. The method of claim 13, wherein said tyrosine protein kinase is Bcr-Abl.

15. A method of inhibiting Bcr-abl activity, the method comprising contacting Bcr-abl with a compound that binds to a myristoyl binding pocket of Bcr-abl.

16. The method of claim 15, wherein the compound is a compound of claim 1.

17. A process for preparing a compound of claim 1, said process comprising:
(a) reacting a compound of Formula 2 with a compound of Formula 3, 4, 5 or 6: 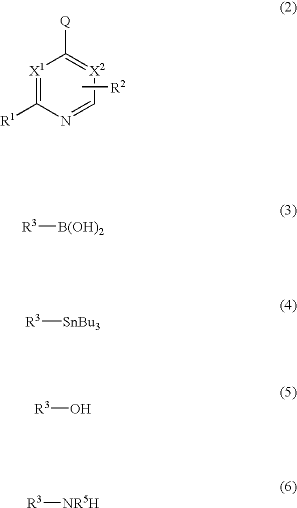
in which X¹, X², R¹, R², R³ and R⁵ are as defined for Formula I above and Q represents a fluoro, chloro, bromo or iodo; or
(b) optionally converting a compound of the invention into a pharmaceutically acceptable salt;
(c) optionally converting a salt form of a compound of the invention to a non-salt form;
(d) optionally converting an unoxidized form of a compound of the invention into a pharmaceutically acceptable N-oxide;
(e) optionally converting an N-oxide form of a compound of the invention to its unoxidized form;
(f) optionally resolving an individual isomer of a compound of the invention from a mixture of isomers;
(g) optionally converting a non-derivatized compound of the invention into a pharmaceutically acceptable prodrug derivative; and
(h) optionally converting a prodrug derivative of a compound of the invention to its non-derivatized form.
